(19) United States
(12) Patent Application Publication
(10) Pub. No.: US 2006/0166917 A1
(43) Pub. Date: Jul. 27, 2006

(54) Differentiation and/or proliferation modulating agents and uses therefor

(75) Inventors: Geoffrey Lindeman, Victoria (AU); Jane Visvader, Victoria (AU)

Correspondence Address: SCULLY, SCOTT, MURPHY & PRESSER;Suite 300, 400 Garden City, Garden City, NY, 11530, US

(73) Assignee: The Walter and Eliza Hall Institute of Medical Research, Victoria (AU)

(21) Appl. No.: 11/265,898

(22) Filed: Nov. 3, 2005

Related U.S. Application Data
(63) Continuation of application No. 10/331,695, filed on Dec. 30, 2002, now abandoned.
(60) Provisional application No. 60/344,152, filed on Dec. 28, 2001.

(51) Int. Cl. A61K 48/00

(52) U.S. Cl. 514/44

(57) ABSTRACT

The present invention discloses methods for modulating the differentiation and/or proliferation of mammary cells, especially of mammary epithelial cells, or for modulating the differentiation and/or proliferation of the lobuloalveolar system, or for modulating mammopoiesis and/or lactogenesis, or for modulating tumorigenesis in a cell which is associated with the reproductive system of a mammal by modulating the expression of a gene or the level and/or functional activity of an expression product of the gene, wherein the gene is selected from SOCS-1 or a gene belonging to the same regulatory or biosynthetic pathway as SOCS-1.

### FIELD OF THE INVENTION

THE INVENTION relates generally to agents that modulate cellular differentiation and/or proliferation. More particularly, the present invention relates to a method of modulating the differentiation and/or proliferation of mammary cells, especially of mammary epithelial cells, by modulating the expression of a gene or the level and/or functional activity of an expression product of the gene, wherein the gene is selected from SOCS-1 or a gene belonging to the same regulatory or biosynthetic pathway as SOCS-1. Even more particularly, the present invention relates to a method of modulating the differentiation and/or proliferation of the lobuloalveolar system by modulating the expression of a gene or the level and/or functional activity of an expression product of the gene, wherein the gene is selected from SOCS-1 or a gene belonging to the same regulatory or biosynthetic pathway as SOCS-1. Still even more particularly, the invention relates to a method for modulating mammopoiesis and/or lactogenesis by modulating the expression of a gene or the level and/or functional activity of an expression product of the gene, wherein the gene is selected from SOCS-1 or a gene belonging to the same regulatory or biosynthetic pathway as SOCS-1. The invention is also concerned with a method of modulating tumorigenesis in a cell which is associated with the reproductive system by modulating the expression of a gene or the level and/or functional activity of an expression product of the gene, wherein the gene is selected from SOCS-1 or a gene belonging to the same regulatory or biosynthetic pathway as SOCS-1. The invention also extends to a method of identifying mammary cell differentiation- and/or proliferation-modulating agents by screening for agents which modulate the expression of a gene or the level and/or functional activity of an expression product of the gene, wherein the gene is selected from SOCS-1 or a gene belonging to the same regulatory or biosynthetic pathway as SOCS-1. The invention is also directed to the use of such modulatory agents in methods for modulating mammary cell differentiation and/or proliferation, for modulating the differentiation and/or proliferation of the lobuloalveolar system, for modulating mammopoiesis and/or lactogenesis in an animal or for enhancing milk yield in a milk producing animal. The invention also encompasses the use of the modulatory agents of the invention for the treatment and/or prophylaxis of cancer, particularly breast cancer.

Bibliographic details of the publications referenced in this specification are collected at the end of the description.

### BACKGROUND OF THE INVENTION

Mammary gland development is governed by the coordinated action of peptide and steroid hormones, such as prolactin, estrogen and progesterone. Prolactin, a pituitary polypeptide hormone, is a key regulator of mammopoiesis (Vonderhaar 1987; Hennighausen et al. 1997). During pregnancy, prolactin is essential for expansion and differentiation of the lobuloalveolar system. After parturition, prolactin acts in synergy with insulin and glucocorticoids, to induce terminal differentiation and milk production. Binding of prolactin to its cognate receptor (PRLR) triggers dimerisation and results in the recruitment and activation of Janus-2 kinase (Jak2). In turn, activated Jak2 phosphorylates the receptor and signal transducer and activator of transcription, Stat5. Activated Stat5 dimers translocate to the nucleus where they lead to transcriptional activation of target genes, including those encoding several milk proteins (Watson and Burdon 1996; Hennighausen et al. 1997; Bole-Feysot et al. 1998).

Targeted disruption of genes in the prolactin signaling pathway has highlighted its importance in mammopoiesis and lactogenesis. Prolactin-deficient mice exhibit curtailed ductal branching with arrest of mammary organogenesis at puberty (Horseman et al. 1997). Interestingly, female mice carrying only one intact prolactin receptor allele fail to lactate after their first pregnancy, demonstrating that differentiation is dependent on a threshold level of PRLR (Ormandy et al. 1997; Brisken et al. 1999). Stat5a-null females show a mammary phenotype similar to that of the PRLR^{+/-} females, exhibiting impaired differentiation of lobuloalveolar units and an inability to lactate (Liu et al. 1997; Teglund et al. 1998).

Although the intracellular signaling pathways activated by prolactin are relatively well understood, the mechanisms by which signaling is attenuated are yet to be defined. Negative regulation is likely to involve protein tyrosine phosphatases as well as specific inhibitory molecules such as the suppressor of cytokine signaling (SOCS) proteins. The SOCS family of proteins appears to act in a classical negative feedback loop to regulate signal transduction by a variety of cytokines (Yoshimura 1998; Krebs and Hilton 2000). The eight members (SOCS-1-7 and CIS) of this family are characterized structurally by a C-terminal SOCS box, a central SH2 domain, and an N-terminal region of variable length and limited homology (Hilton et al. 1998). Functionally, SOCS proteins interact with cytokine receptors and/or Jak kinases, thereby inhibiting activation of kinases and STAT proteins (Yoshimura 1998; Krebs and Hilton 2000).

SOCS-1, one of the founding members of the SOCS family (also termed JAB or SSI-1) (Endo et al. 1997; Naka et al. 1997; Starr et al. 1997), is induced in response to a broad range of cytokines and interacts with the kinase domain of Jak proteins. SOCS-1-deficient mice die from a complex neonatal disease prior to weaning, involving fatty degeneration of the liver and multiple hematopoietic defects (Naka et al. 1998; Starr et al. 1998). This multiorgan disease can be prevented by neonatal treatment with neutralizing anti-interferon gamma (IFNγ) antibodies and is absent in mice lacking both SOCS-1 and IFNγ genes, indicating that SOCS-1 is a key modulator of IFNγ effects (Alexander et al. 1999; Marine et al. 1999a). Thus, additional disruption of the IFNγ gene allows the effects of SOCS-1-gene deficiency to be studied in adult mice.

In work leading up to the present invention, the inventors studied mice carrying targeted deletions of the SOCS-1 and IFNγ genes to investigate the role of SOCS-1 in the mammary gland. Surprisingly, these mice exhibited accelerated lobuloalveolar development during pregnancy. Moreover, deletion of a single copy of SOCS-1 rescued the lactogenic defect that occurs in PRLR^{+/-} mice (Ormandy et al. 1997). These findings provide evidence that SOCS-1 has a biological role in the developing mammary gland, where it acts as a negative regulator of prolactin signaling. Further, the data demonstrate that the absolute levels of both positive and negative modulators of the prolactin pathway are critical for directing expansion and differentiation of the mammary gland. The present inventors have also found that SOCS-1 deficiency in female mice is associated with a higher incidence of breast and ovarian carcinomas. In this light, and given that SOCS genes are potent inhibitors of multiple cytokine signalling pathways, the present inventors believe that SOCS-1 is a tumour suppressor in tissues of the reproductive organs, especially in breast and ovarian tissues. It is proposed, therefore, that SOCS-1 or its expression products can be used inter alia to provide both drug targets and regulators to promote or inhibit mammopoiesis and/or lactogenesis, or to abrogate or reduce tumorigenesis, as described hereinafter.

### SUMMARY OF THE INVENTION

Accordingly, in one aspect of the present invention, there is provided a method for modulating the differentiation of a mammary cell, comprising modulating in the mammary cell the expression of a gene or the level and/or functional activity of an expression product of the gene, wherein the gene is selected from SOCS-1 or a gene belonging to the same regulatory or biosynthetic pathway as SOCS-1.

In a related aspect, the present invention envisions a method for modulating the differentiation of a mammary cell, comprising modulating in the mammary cell the expression of SOCS-1 or the level and/or functional activity of an expression product of SOCS-1.

In yet another aspect, the present invention features a method for modulating the proliferation of a mammary cell, comprising modulating in the mammary cell the expression of a gene or the level and/or functional activity of an expression product of the gene, wherein the gene is selected from SOCS-1 or a gene belonging to the same regulatory or biosynthetic pathway as SOCS-1.

In a related aspect, the present invention resides in a method for modulating the proliferation of a mammary cell, comprising modulating in the mammary cell the expression of SOCS-1 or the level and/or functional activity of an expression product of SOCS-1.

In still yet another aspect, the present invention provides a method for modulating the differentiation and proliferation of a mammary cell, comprising modulating in the mammary cell the expression of a gene or the level and/or functional activity of an expression product of the gene, wherein the gene is selected from SOCS-1 or a gene belonging to the same regulatory or biosynthetic pathway as SOCS-1.

In a related aspect, the present invention contemplates a method for modulating the differentiation and proliferation of a mammary cell, comprising modulating in the mammary cell the expression of SOCS-1 or the level and/or functional activity of an expression product of SOCS-1.

In another aspect, the present invention encompasses a method for modulating mammopoiesis, comprising modulating in a mammary cell the expression of a gene or the level and/or functional activity of an expression product of the gene, wherein the gene is selected from SOCS-1 or a gene belonging to the same regulatory or biosynthetic pathway as SOCS-1.

In a related aspect, the present invention contemplates a method for modulating mammopoiesis, comprising modulating in a mammary cell the expression of SOCS-1 or the level and/or functional activity of an expression product of SOCS-1.

In a further aspect, the present invention encompasses a method for modulating the differentiation of the lobuloalveolar system, comprising modulating the expression of a gene or the level and/or functional activity of an expression product of the gene in a mammary cell, wherein the gene is selected from SOCS-1 or a gene belonging to the same regulatory or biosynthetic pathway as SOCS-1.

In a related aspect, the present invention envisions a method for modulating the differentiation of the lobuloalveolar system, comprising modulating in a mammary cell the expression of SOCS-1 or the level and/or functional activity of an expression product of SOCS-1.

In yet a further aspect, the present invention features a method for modulating the expansion of the lobuloalveolar system, comprising modulating the expression of a gene or the level and/or functional activity of an expression product of the gene in a mammary cell, wherein the gene is selected from SOCS-1 or a gene belonging to the same regulatory or biosynthetic pathway as SOCS-1.

In a related aspect, the present invention resides in a method for modulating the expansion of the lobuloalveolar system, comprising modulating in a mammary cell the expression of SOCS-1 or the level and/or functional activity of an expression product of SOCS-1.

In still yet a further aspect, the present invention provides a method for modulating the differentiation and expansion of the lobuloalveolar system, comprising modulating the expression of a gene or the level and/or functional activity of an expression product of the gene in a mammary cell, wherein the gene is selected from SOCS-1 or a gene belonging to the same regulatory or biosynthetic pathway as SOCS-1.

In a related aspect, the present invention resides in a method for modulating the differentiation and expansion of the lobuloalveolar system, comprising modulating in a mammary cell the expression of SOCS-1 or the level and/or functional activity of an expression product of SOCS-1.

In another aspect, the present invention provides a method for modulating lactogenesis, comprising modulating the expression of a gene or the level and/or functional activity of an expression product of the gene in a mammary cell, wherein the gene is selected from SOCS-1 or a gene belonging to the same regulatory or biosynthetic pathway as SOCS-1.

In a related aspect, the present invention contemplates a method for modulating lactogenesis, comprising modulating in a mammary cell the expression of SOCS-1 or the level and/or functional activity of an expression product of SOCS-1.

In preferred embodiments of the methods broadly described above, the expression of the gene or the level and/or functional activity of the expression product is abrogated or reduced.

In other preferred embodiments of the methods broadly described above, the mammary cell is a mammary epithelial cell. Suitably, the mammary epithelial cell is a mammary ductal epithelial cell.

In yet another aspect, the present invention encompasses a method for modulating tumorigenesis in a cell that is associated with the reproductive system of a mammal, comprising modulating the expression of a gene or the level and/or functional activity of an expression product of the gene, wherein the gene is selected from SOCS-1 or a gene belonging to the same regulatory or biosynthetic pathway as SOCS-1.

In a related aspect, the present invention envisions a method for modulating tumorigenesis in a cell that is associated with the reproductive system of a mammal, comprising modulating the expression of SOCS-1 or the level and/or functional activity of an expression product of SOCS-1.

In a preferred embodiment of this type, the expression of the gene or the level and/or functional activity of the expression product is enhanced. The cell is preferably a cell of a reproductive organ selected from breast, ovary, endometrium, testes, and prostate. More preferably, the cell is a mammary cell.

In another aspect, the present invention encompasses a method for modulating the differentiation of a mammary cell, comprising contacting the mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention envisions a method for modulating the differentiation of a mammary cell, comprising contacting the mammary cell with an agent for a time and under conditions sufficient to modulate the expression of SOCS-1 or the level and/or functional activity of an expression product of SOCS-1.

In yet another aspect, the present invention features a method for modulating the proliferation of a mammary cell, comprising contacting the mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention resides in a method for modulating the proliferation of a mammary cell, comprising contacting the mammary cell with an agent for a time and under conditions sufficient to modulate the expression of SOCS-1 or the level and/or functional activity of an expression product of SOCS-1.

In still yet another aspect, the present invention provides a method for modulating the differentiation and proliferation of a mammary cell, comprising contacting the mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention contemplates a method for modulating the differentiation and proliferation of a mammary cell, comprising contacting the mammary cell with an agent for a time and under conditions sufficient to modulate the expression of SOCS-1 or the level and/or functional activity of an expression product of SOCS-1.

In yet another aspect, the invention contemplates a method for modulating mammopoiesis, comprising contacting a mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention contemplates a method for modulating mammopoiesis, comprising contacting a mammary cell with an agent for a time and under conditions sufficient to modulate the expression of SOCS-1 or the level and/or functional activity of an expression product of SOCS-1.

In a further aspect, the present invention encompasses a method for modulating the differentiation of the lobuloalveolar system, comprising contacting a mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention envisions a method for modulating the differentiation of the lobuloalveolar system, comprising contacting a mammary cell with an agent for a time and under conditions sufficient to modulate the expression of SOCS-1 or the level and/or functional activity of an expression product of SOCS-1.

In yet a further aspect, the present invention features a method for modulating the expansion of the lobuloalveolar system, comprising contacting a mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention resides in a method for modulating the expansion of the lobuloalveolar system, comprising contacting a mammary cell with an agent for a time and under conditions sufficient to modulate the expression of SOCS-1 or the level and/or functional activity of an expression product of SOCS-1.

In still yet a further aspect, the present invention provides a method for modulating the differentiation and expansion of the lobuloalveolar system, comprising contacting a mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In still yet a further aspect, the present invention provides a method for modulating the differentiation and expansion of the lobuloalveolar system, comprising contacting a mammary cell with an agent for a time and under conditions sufficient to modulate the expression of SOCS-1 or the level and/or functional activity of an expression product of SOCS-1.

In another aspect, the present invention provides a method for modulating lactogenesis, comprising contacting a mammary cell with an agent for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention contemplates a method for modulating lactogenesis, comprising contacting a mammary cell with an agent for a time and under conditions sufficient to modulate the expression of SOCS-1 or the level and/or functional activity of an expression product of SOCS-1.

In yet another aspect, the present invention encompasses a method for modulating tumorigenesis in a cell that is associated with the reproductive system of a mammal, comprising contacting the cell for a time and under conditions sufficient to modulate the expression of a gene or the level and/or functional activity of an expression product of the gene, wherein the gene is selected from SOCS-1 or a gene belonging to the same regulatory or biosynthetic pathway as SOCS-1.

In a related aspect, the present invention envisions a method for modulating tumorigenesis in a cell that is associated with the reproductive system of a mammal, comprising contacting the cell for a time and under conditions sufficient to modulate the expression of SOCS-1 or the level and/or functional activity of an expression product of SOCS-1.

Still another aspect of the invention contemplates the use of an agent which modulates the expression of a gene or the level and/or functional activity of an expression product of the gene, wherein the gene is selected from SOCS-1 or a gene belonging to the same regulatory or biosynthetic pathway as SOCS-1, in the preparation of a medicament for modulating differentiation of a mammary cell, for modulating proliferation of a mammary cell, for modulating differentiation and proliferation of a mammary cell, for modulating mammopoiesis, for modulating the differentiation of the lobuloalveolar system, for modulating the expansion of the lobuloalveolar system or for modulating lactogenesis.

In a related aspect, the invention encompasses the use of an agent which modulates the expression of SOCS-1 or the level and/or functional activity of an expression product of SOCS-1 in the preparation of a medicament for modulating differentiation of a mammary cell, for modulating proliferation of a mammary cell, for modulating differentiation and proliferation of a mammary cell, for modulating mammopoiesis, for modulating the differentiation of the lobuloalveolar system, for modulating the expansion of the lobuloalveolar system or for modulating lactogenesis.

In another aspect, the invention contemplates the use of an agent which modulates the expression of a gene or the level and/or functional activity of an expression product of the gene, wherein the gene is selected from SOCS-1 or a gene belonging to the same regulatory or biosynthetic pathway as SOCS-1, in the preparation of a medicament for modulating tumorigenesis in a cell that is associated with the reproductive system of a mammal.

In a related aspect, the invention encompasses the use of an agent which modulates the expression of SOCS-1 or the level and/or functional activity of an expression product of SOCS-1 in the preparation of a medicament for modulating differentiation of a mammary cell, for modulating tumorigenesis in a cell that is associated with the reproductive system of a mammal.

In still yet another aspect, the invention provides a composition for potentiating or promoting differentiation of a mammary cell, comprising an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention features a composition for potentiating or promoting differentiation of a mammary cell, comprising an agent which inhibits, abrogates or otherwise reduces the expression of SOCS-1 or the level and/or functional activity of an expression product of SOCS-1, together with a pharmaceutically acceptable carrier and/or diluent.

In another aspect, the invention envisions a composition for potentiating or promoting proliferation of a mammary cell, comprising an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention encompasses a composition for potentiating or promoting proliferation of a mammary cell, comprising an agent which inhibits, abrogates or otherwise reduces the expression of SOCS-1 or the level and/or functional activity of an expression product of SOCS-1, together with a pharmaceutically acceptable carrier and/or diluent.

In another aspect, the invention contemplates a composition for potentiating or promoting mammopoiesis, comprising an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention encompasses a composition for potentiating or promoting mammopoiesis, comprising an agent which inhibits, abrogates or otherwise reduces the expression of SOCS-1 or the level and/or functional activity of an expression product of SOCS-1, together with a pharmaceutically acceptable carrier and/or diluent.

In another aspect, the invention provides a composition for potentiating or promoting differentiation of the lobuloalveolar system, comprising an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention features a composition for potentiating or promoting differentiation of the lobuloalveolar system, comprising an agent which inhibits, abrogates or otherwise reduces the expression of SOCS-1 or the level and/or functional activity of an expression product of SOCS-1, together with a pharmaceutically acceptable carrier and/or diluent.

In still another aspect, the invention resides in a composition for potentiating or promoting expansion of the lobuloalveolar system, comprising an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention features a composition for potentiating or promoting expansion of the lobuloalveolar system, comprising an agent which inhibits, abrogates or otherwise reduces the expression of SOCS-1 or the level and/or functional activity of an expression product of SOCS-1, together with a pharmaceutically acceptable carrier and/or diluent.

In a further aspect, the invention contemplates a composition for potentiating or promoting lactogenesis, comprising an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention envisions a composition for potentiating or promoting lactogenesis, comprising an agent which inhibits, abrogates or otherwise reduces the expression of SOCS-1 or the level and/or functional activity of an expression product of SOCS-1, together with a pharmaceutically acceptable carrier and/or diluent.

In another aspect, the invention extends to a composition for the treatment and/or prophylaxis of a cancer of a reproductive organ, comprising an agent which enhances the expression of a gene or the level and/or functional activity of an expression product of the gene, wherein the gene is selected from SOCS-1 or a gene belonging to the same regulatory or biosynthetic pathway as SOCS-1, together with a pharmaceutically acceptable carrier and/or diluent.

In a related aspect, the invention features a composition for the treatment and/or prophylaxis of a cancer of a reproductive organ, comprising an agent which enhances the expression of SOCS-1 or the level and/or functional activity of an expression product of SOCS-1, together with a pharmaceutically acceptable carrier and/or diluent.

According to another aspect of the invention, there is provided a method for potentiating or promoting mammopoiesis in an animal, comprising administering to the animal a mammopoiesis-potentiating or -promoting effective amount of an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1.

In a related aspect, the invention contemplates a method for potentiating or promoting mammopoiesis in an animal, comprising administering to the animal a mammopoiesis-potentiating or -promoting effective amount of an agent which inhibits, abrogates or otherwise reduces the expression of SOCS-1 or the level and/or functional activity of an expression product of SOCS-1.

In another aspect, the invention encompasses a method for potentiating or promoting lactogenesis in an animal, comprising administering to the animal a lactogenesis-potentiating or -promoting effective amount of an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1.

In a related aspect, the invention contemplates a method for potentiating or promoting lactogenesis in an animal, comprising administering to the animal a lactogenesis-potentiating or -promoting effective amount of an agent which inhibits, abrogates or otherwise reduces the expression of SOCS-1 or the level and/or functional activity of an expression product of SOCS-1.

In yet another aspect, the invention provides a method for the treatment and/or prophylaxis of breast cancer or a related condition, comprising administering to a patient in need of such treatment a symptom-ameliorating effective amount of an agent which enhances a gene or the level and/or functional activity of an expression product of the gene, wherein the gene is selected from SOCS-1 or a gene belonging to the same regulatory or biosynthetic pathway as SOCS-1.

In a related aspect, the invention extends to a method for the treatment and/or prophylaxis of breast cancer or a related condition, comprising administering to a patient in need of such treatment a symptom-ameliorating effective amount of an agent which enhances the expression of SOCS-1 or the level and/or functional activity of an expression product of SOCS-1.

In another aspect, the invention encompasses a method for potentiating, promoting or otherwise enhancing milk production in a milk producing animal, comprising administering to the animal a milk production-potentiating, -promoting or otherwise-enhancing effective amount of an agent which inhibits, abrogates or otherwise reduces the expression of a gene or the level and/or functional activity of an expression product of the gene, wherein the gene is selected from SOCS-1 or a gene belonging to the same regulatory or biosynthetic pathway as SOCS-1.

In another aspect, the invention contemplates a method for potentiating, promoting or otherwise enhancing milk production in a milk producing animal, comprising administering to the animal a milk production-potentiating, -promoting or otherwise-enhancing effective amount of an agent which inhibits, abrogates or otherwise reduces the expression of SOCS-1 or the level and/or functional activity of an expression product of SOCS-1.

In yet another aspect, the invention extends to a method of screening for an agent which modulates the differentiation of a mammary cell, comprising:
contacting a preparation comprising a SOCS-1 polypeptide or biologically active fragment thereof, or variant or derivative of these, or a genetic sequence that modulates the expression of SOCS-1, with a test agent; and
detecting a change in the level and/or functional activity of the SOCS-1 polypeptide or biologically active fragment thereof, or variant or derivative, or of a product expressed from the genetic sequence.

In still yet another aspect, the invention provides a method of screening for an agent which potentiates or promotes the differentiation of a mammary cell, comprising:
contacting a preparation comprising a SOCS-1 polypeptide or biologically active fragment thereof, or variant or derivative of these, or a genetic sequence that modulates the expression of SOCS-1, with a test agent; and
detecting inhibition, abrogation or otherwise reduction in the level and/or functional activity of the SOCS-1 polypeptide or biologically active fragment thereof, or variant or derivative, or of a product expressed from the genetic sequence.

In another aspect, the invention envisions a method of screening for an agent which modulates proliferation of a mammary cell, comprising:
contacting a preparation comprising a SOCS-1 polypeptide or biologically active fragment thereof, or variant or derivative of these, or a genetic sequence that modulates the expression of SOCS-1, with a test agent; and
detecting a change in the level and/or functional activity of the SOCS-1 polypeptide or biologically active fragment thereof, or variant or derivative, or of a product expressed from the genetic sequence.

In yet another aspect, the invention envisions a method of screening for an agent which potentiates or promotes the proliferation of a mammary cell, comprising:
contacting a preparation comprising a SOCS-1 polypeptide or biologically active fragment thereof, or variant or derivative of these, or a genetic sequence that modulates the expression of SOCS-1, with a test agent; and
detecting inhibition, abrogation or otherwise reduction in the level and/or functional activity of the SOCS-1 polypeptide or biologically active fragment thereof, or variant or derivative, or of a product expressed from the genetic sequence.

In another aspect, the invention contemplates a method of screening for an agent which modulates mammopoiesis, comprising:
contacting a preparation comprising a SOCS-1 polypeptide or biologically active fragment thereof, or variant or derivative of these, or a genetic sequence that modulates the expression of SOCS-1, with a test agent; and
detecting a change in the level and/or functional activity of the SOCS-1 polypeptide or biologically active fragment thereof, or variant or derivative, or of a product expressed from the genetic sequence.

In a further aspect, the invention features a method of screening for an agent which potentiates or promotes mammopoiesis, comprising:
contacting a preparation comprising a SOCS-1 polypeptide or biologically active fragment thereof, or variant or derivative of these, or a genetic sequence that modulates the expression of SOCS-1, with a test agent; and
detecting inhibition, abrogation or otherwise reduction in the level and/or functional activity of the SOCS-1 polypeptide or biologically active fragment thereof, or variant or derivative, or of a product expressed from the genetic sequence.

In still a further aspect, the invention encompasses a method of screening for an agent which modulates the differentiation of the lobuloalveolar system, comprising:
contacting a preparation comprising a SOCS-1 polypeptide or biologically active fragment thereof, or variant or derivative of these, or a genetic sequence that modulates the expression of SOCS-1, with a test agent; and
detecting a change in the level and/or functional activity of the SOCS-1 polypeptide or biologically active fragment thereof, or variant or derivative, or of a product expressed from the genetic sequence.

In another aspect, the invention features a method of screening for an agent which potentiates or promotes the differentiation of the lobuloalveolar system, comprising:
contacting a preparation comprising a SOCS-1 polypeptide or biologically active fragment thereof, or variant or derivative of these, or a genetic sequence that modulates the expression of SOCS-1, with a test agent; and
detecting inhibition, abrogation or otherwise reduction in the level and/or functional activity of the SOCS-1 polypeptide or biologically active fragment thereof, or variant or derivative, or of a product expressed from the genetic sequence.

In yet another aspect, the invention contemplates a method of screening for an agent which modulates the expansion of the lobuloalveolar system, comprising:
contacting a preparation comprising a SOCS-1 polypeptide or biologically active fragment thereof, or variant or derivative of these, or a genetic sequence that modulates the expression of SOCS-1, with a test agent; and
detecting a change in the level and/or functional activity of the SOCS-1 polypeptide or biologically active fragment thereof, or variant or derivative, or of a product expressed from the genetic sequence.

In still yet another aspect, the invention envisions a method of screening for an agent which potentiates or promotes the expansion of the lobuloalveolar system, comprising:
contacting a preparation comprising a SOCS-1 polypeptide or biologically active fragment thereof, or variant or derivative of these, or a genetic sequence that modulates the expression of SOCS-1, with a test agent; and
detecting inhibition, abrogation or otherwise reduction in the level and/or functional activity of the SOCS-1 polypeptide or biologically active fragment thereof, or variant or derivative, or of a product expressed from the genetic sequence.

In yet another aspect, the invention extends to a method of screening for an agent which modulates lactogenesis, comprising:
contacting a preparation comprising a SOCS-1 polypeptide or biologically active fragment thereof, or variant or derivative of these, or a genetic sequence that modulates the expression of SOCS-1, with a test agent; and
detecting a change in the level and/or functional activity of the SOCS-1 polypeptide or biologically active fragment thereof, or variant or derivative, or of a product expressed from the genetic sequence.

In still yet another aspect, the invention provides a method of screening for an agent which potentiates or promotes lactogenesis, comprising:
contacting a preparation comprising a SOCS-1 polypeptide or biologically active fragment thereof, or variant or derivative of these, or a genetic sequence that modulates the expression of SOCS-1, with a test agent; and
detecting inhibition, abrogation or otherwise reduction in the level and/or functional activity of the SOCS-1 polypeptide or biologically active fragment thereof, or variant or derivative, or of a product expressed from the genetic sequence.

In a preferred embodiment of the above screening methods, the fragment comprises the SH2 domain of the SOCS-1 polypeptide.

In another preferred embodiment of the above screening methods, the fragment comprises the SOCS box motif of the SOCS-1 polypeptide.

### BRIEF DESCRIPTION OF THE DRAWINGS

FIG. 1 is a photographic representation of an H&E section of an adnexal mass showing adenocarcinoma cells forming pseudoducts (arrows) surrounded by chronic inflammatory cells (Orig mag. 20×).

FIG. 2 is a photographic representation of an H&E section showing a hypercellular, multi-layered duct, with nuclear atypia. Numerous apoptotic bodies are present (arrows). (Normal ducts contains two cell layers) (Orig mag. 200×).

### BRIEF DESCRIPTION OF THE SEQUENCES: SUMMARY TABLE

TABLE A

| SEQUENCE ID | | | |
|---|---|---|---|
| NUMBER | SEQUENCE | LENGTH | |
| SEQ ID NO: 1 | Human SOCS-1 cDNA | 908 | nts |
| SEQ ID NO: 2 | Human SOCS-1 polypeptide | 211 | aa |
| SEQ ID NO: 3 | Mouse SOCS-1 cDNA | 1193 | nts |
| SEQ ID NO: 4 | Mouse SOCS-1 polypeptide | 212 | aa |
| SEQ ID NO: 5 | Human SH2 domain | 63 | aa |
| SEQ ID NO: 6 | Mouse SH2 domain | 63 | aa |
| SEQ ID NO: 7 | Human extended SH2 domain | 104 | aa |
| SEQ ID NO: 8 | Mouse extended SH2 domain | 104 | aa |
| SEQ ID NO: 9 | Human SOCS-1 SOCS box | 34 | aa |
| SEQ ID NO: 10 | Mouse SOCS-1 SOCS box | 34 | aa |
| SEQ ID NO: 11 | Human extended SOCS-1 SOCS box | 43 | aa |
| SEQ ID NO: 12 | Mouse extended SOCS-1 SOCS box | 43 | aa |
| SEQ ID NO: 13 | β-casein sense primer | 25 | nts |
| SEQ ID NO: 14 | β-casein antisense primer | 26 | nts |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and nonimmunoglobulin derived protein frameworks that exhibit antigen-binding activity.

“Antigenic or immunogenic activity” refers to the ability of a polypeptide, fragment, variant or derivative according to the invention to produce an antigenic or immunogenic response in an animal, preferably a mammal, to which it is administered, wherein the response includes the production of elements which specifically bind the polypeptide or fragment thereof.

By “biologically active fragment” is meant a fragment of a full-length parent polypeptide which fragment retains the activity of the parent polypeptide. As used herein, the term “biologically active fragment” includes deletion variants and small peptides, for example of at least 10, preferably at least 20 and more preferably at least 30 contiguous amino acids, which comprise the above activities. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “complementary” refers to the topological capability or matching together of interacting surfaces of a test polynucleotide and its target oligonucleotide, which may be part of a larger polynucleotide. Thus, the test and target polynucleotides can be described as complementary, and furthermore, the contact surface characteristics are complementary to each other. Complementary includes base complementarity such as A is complementary to T or U and C is complementary to G in the genetic code. However, this invention also encompasses situations in which there is non-traditional base-pairing such as Hoogsteen base pairing which has been identified in certain transfer RNA molecules and postulated to exist in a triple helix. In the context of the definition of the term “complementary”, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that hybridise less efficiently.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions or deletions that provide for functional equivalent molecules.

By “effective amount”, in the context of modulating an activity or of treating or preventing a condition is meant the administration of that amount of active to an individual in need of such modulation, treatment or prophylaxis, either in a single dose or as part of a series, that is effective for modulation of that effect or for treatment or prophylaxis of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “function” refers to a biological, enzymatic, or therapeutic function.

“Homology” refers to the percentage number of amino acids that are identical or constitute conservative substitutions as defined in Table B below. Homology may be determined using sequence comparison programs such as GAP (Deveraux et al 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

As used herein the term “level” refers to a concentration or amount of a molecule or substance or group of molecules or substances in a cell or sample.

By “modulating” is meant increasing or decreasing, either directly or indirectly, the level and/or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a polynucleotide extract or polypeptide extract is isolated from, or derived from, a particular source of the host. For example, the extract can be obtained from a tissue or a biological fluid isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant that transcriptional and translational regulatory polynucleotides are positioned relative to a polypeptide-encoding polynucleotide in such a manner that the polynucleotide is transcribed and the polypeptide is translated.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

By “pharmaceutically acceptable carrier” is meant a solid or liquid filler, diluent or encapsulating substance that can be safely used in topical or systemic administration to a mammal.

The term “polynucleotide” or “nucleic acid” as used herein designates mRNA, RNA, cRNA, cDNA or DNA. The term typically refers to oligonucleotides greater than 30 nucleotide residues in length.

The terms “polynucleotide variant” and “variant” refer to polynucleotides displaying substantial sequence identity with a reference polynucleotide sequence or polynucleotides that hybridize with a reference sequence under stringent conditions that are defined hereinafter. These terms also encompasses polynucleotides which differ from a reference polynucleotide by the addition, deletion or substitution of at least one nucleotide. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides which differ from a reference polypeptide by the addition, deletion or substitution of at least one amino acid. It is well understood in the art for example that some amino acids may be changed to others with broadly similar properties without changing the nature of the activity of the polypeptide (conservative substitutions) as described hereinafter.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, i.e., through the expression of a recombinant or synthetic polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

The term “reproductive system” refers to the bodily system of gonads, associated ducts, and external genitals concerned with sexual reproduction, including the testis, ovary, endometrium, prostate, uterus, cervix and breast.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridization. The higher the stringency, the higher will be the degree of complementarity between immobilized polynucleotides and the labeled polynucleotide.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing.

“Stringent conditions” refers to temperature and ionic conditions under which only nucleotide sequences having a high frequency of complementary bases will hybridize. The stringency required is nucleotide sequence dependent and depends upon the various components present during hybridization and subsequent washes, and the time allowed for these processes. Generally, in order to maximize the hybridization rate, non-stringent hybridization conditions are selected; about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately stringent washing conditions are selected to be about 15 to 30° C. lower than the Tₘ. Highly permissive (low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

The terms “wild-type” and “normal” are used interchangeably to refer to the phenotype that is characteristic of most of the members of the species occurring naturally and contrast for example with the phenotype of a mutant.

As used herein, underscoring or italicizing the name of a gene shall indicate the gene, in contrast to its protein product, which is indicated by the name of the gene in the absence of any underscoring or italicizing. For example, “SOCS-1” shall mean the SOCS-1 gene, whereas “SOCS” shall indicate the protein product of the “SOCS-1” gene.

2. Method of Modulating Mammopoiesis and Lactogenesis

The present invention is predicated in part on the determination that SOCS-1 deficiency in mice results in accelerated mammary gland development and rescues lactation in prolactin receptor deficient mice. It is believed, therefore, that SOCS-1 is a negative regulator of prolactin signaling and suggests that SOCS-1 is required for the prevention of lactation prior to parturition. Accordingly, it is proposed that modulators of SOCS-1 polynucleotides or SOCS-1 polypeptides will be useful inter alia in the modulation of differentiation and/or proliferation of mammary cells and in the modulation of mammopoiesis and lactogenesis.

Thus, in one aspect of the present invention, there is broadly provided a method for modulating the differentiation and/or proliferation of a mammary cell, comprising modulating the expression of a gene or the level and/or functional activity of an expression product of the gene in the mammary cell, wherein the gene is selected from SOCS-1 or a gene belonging to the same regulatory or biosynthetic pathway as SOCS-1. In a related embodiment, the gene expression or the level and/or functional activity of the expression product is modulated to regulate or control mammopoiesis and/or lactogenesis. In another related embodiment, the gene expression or the level and/or functional activity of the expression product is modulated to regulate or control the differentiation and/or proliferation of the lobuloalveolar system.

The gene belonging to the same regulatory or biosynthetic pathway as SOCS-1 suitably encodes an expression product that modulates the expression of SOCS-1 or the level and/or functional activity of an expression product of SOCS-1. Alternatively, that gene or its expression product may be modulated by an expression product of SOCS-1 and may comprise, for example, a downstream cellular target of a SOCS-1 expression product.

Preferably, the mammary cell is a mammary epithelial cell, more preferably a mammary ductal epithelial cell.

In one embodiment, the modulation is effected by contacting a mammary cell with an agent that inhibits, abrogates o otherwise reduces the expression of the gene or the level and/or functional activity of an expression product of the gene. Agents that may be used to reduce or abrogate gene expression include, but are not restricted to, oligoribonucleotide sequences, including anti-sense RNA and DNA molecules and ribozymes, that function to inhibit the translation, for example, of SOCS-1-encoding mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of an SOCS-1 gene, are preferred.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of SOCS-1 RNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Both anti-sense RNA and DNA molecules and ribozymes may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to nucleic acid molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioates or 2′ O-methyl rather than phosphodiesterase linkages within the oligodeoxyribonucleotide backbone.

Other agents that may be used to decrease the expression of a gene or the level and/or functional activity of an expression product of that gene include RNA molecules that mediate RNA interference (RNAi) of a target gene or gene transcript. RNAi refers to interference with or destruction of the product of a target gene by introducing a single stranded, and typically a double stranded RNA (dsRNA), which is homologous to the transcript of a target gene. Thus, in one embodiment, dsRNA per se and especially dsRNA-producing constructs corresponding to at least a portion of a SOCS-1 protein may be used to decrease its level and/or functional activity. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a target gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are preferably at least about 100 nucleotides in length. RNA-encoding constructs that express a single RNA designed to form a dsRNA via internal folding are preferably at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors.

In another embodiment, RNA molecules of about 21 to about 23 nucleotides, which direct cleavage of specific mRNA to which they correspond, as for example described by Tuschl et al. in U.S. Patent Application No. 20020086356, can be utilised for mediating RNAi. Such 21-23 nt RNA molecules can comprise a 3′ hydroxyl group, can be single-stranded or double stranded (as two 21-23 nt RNAs) wherein the dsRNA molecules can be blunt ended or comprise overhanging ends (e.g., 5′, 3′).

The present invention also contemplates the use in the above method of gene or expression product inhibitors identified according to methods described for example in Section 4, infra.

In another embodiment, the modulation is effected by contacting a mammary cell with an agent that increases the expression of the gene or the level and/or functional activity of the expression product. Any suitable SOCS-1 inducers or stabilizing/activating agents may be used in this regard and these can be identified by methods disclosed for example in Section 4 infra. In this instance, the agent is suitably used to inhibit mammary cell differentiation and/or proliferation including, for example, inhibiting or abrogating mammopoiesis and/or lactogenesis. An agent that increases the expression of the gene or the level and/or functional activity of the expression product may comprise a SOCS-1 polynucleotide or a SOCS-1 polypeptide. For example, the SOCS-1 polynucleotide comprises a sequence selected from SEQ ID NO: 1 or 3, or genomic sequences relating thereto, or biologically active fragments thereof, or variants of these. Exemplary SOCS-1 polypeptides comprise an amino acid sequence selected from SEQ ID NO: 2 or 4 or biologically active fragments thereof, or variants or derivatives, including mimetics, of these.

The modulatory agents of the invention will suitably affect or modulate the differentiation and/or proliferation of mammary cells, especially the differentiation and/or proliferation of the lobuloalveolar system. Accordingly, the cell that is the subject of testing is preferably a mammary cell, more preferably a mammary epithelial cell and still more preferably a ductal mammary epithelial cell, or progenitor thereof. Suitable assays for testing the effects of modulatory agents on mammary cells include, but are not restricted to, mammary cell proliferation or differentiation assays. The ability of modulatory agents to stimulate or inhibit differentiation or proliferation of mammary cells can be measured using cultured mammary cells, especially mammary epithelial cells, or in vivo by administering molecules of the present invention to the appropriate animal model. Cultured mammary cells include, but are not limited to, normal mammary epithelial cell lines such as MAC-T cells (U.S. Pat. No. 5,227,301), 184A1 cells (Yaswen et al., 1990, Proc. Natl. Acad. Sc. USA 87: 7360-7364), MCF-10A cells (Soule et al., 1990, Cancer Research 50: 6075-6086), HME87 (Gazdar et al., 1998, Int. J. Cancer 78: 766-774), SCp2 cells (Desprez et al., 1993, Mol. Cell Diff. 1: 99-110) and normal rat mammary epithelial cells (Darcy et al., 1991, Exp. Cell Res. 196: 49-65), as well as human mammary carcinoma cell lines BCA-1, ZR-75-1, T-47D, MDA-MB-453, BT-474, H3396, MCF-7, MDA-MB-330, MDB-MB-231, MDA-MB-157, MDA-MB-468, SK-BR-3 and MDA-MB 361. Assays that measure differentiation include, for example, measuring cell-surface markers associated with stage-specific expression of a tissue, enzymatic activity, functional activity or morphological changes (Watt, 1991, FASEB 5: 281-4; Francis, 1994, Differentiation 57: 63-75; Raes, 1989, Adv. Anim. Cell Biol. Technol. Bioprocesses, 161-71). Assays measuring cell proliferation or differentiation are well known in the art. For example, assays measuring proliferation include such assays as chemosensitivity to neutral red dye (Cavanaugh et al., 1990, Investigational New Drugs 8: 347-354), incorporation of radiolabelled nucleotides (Cook et al., 1989, Anal. Biochem. 179: 1-7), incorporation of 5-bromo-2′-deoxyuridine (BrdU) in the DNA of proliferating cells (Porstmann et al., 1985, J. Immunol. Methods 82: 169-79), and use of tetrazolium salts (Mosmann, 1983, J. Immunol. Methods 65 55-63; Alley et al., 1988, Cancer Res. 48: 589-601; Marshall et al., 1995, Growth Reg. 5: 69-84; and Scudiero et al., Cancer Res. 1988, 48: 4827-33) and by measuring proliferation using ³H-thymidine uptake (Crowley et al., 1990, J. Immunol. Meth. 133: 55-66).

In vivo assays, well known in the art, are available for evaluating the effect of SOCS-1/SOCS-1 modulatory agents on the mammary gland. For example, compounds can be injected intraperitoneally or by ductal cannulation for a specific time duration. After the treatment period, animals are sacrificed and mammary glands removed and weighed. Mammary glands are examined by wholemount analysis and histological sectioning to assess the development of lobuloalveolar units and to measure the levels of milk proteins in the glands.

3. Method of Modulating Tumorigenesis in Cells of the Reproductive System

The present inventors have also found that SOCS-1 deficiency in female mice is associated with a higher incidence of breast and ovarian carcinomas. In this light, and because SOCS genes are known to be potent inhibitors of multiple cytokine signalling pathways, the present inventors consider that SOCS-1 is a tumour suppressor in breast and ovarian tissues as well as in tissues of other reproductive organs including the endometrium, testes and prostate. Not wishing to be bound by any one particular theory or mode of operation, the present inventors propose that loss of SOCS-1 leads to inappropriate growth and subsequent tumour formation as a result of continuous signalling of the prolactin pathway. Accordingly, in another aspect, the invention contemplates a method for modulating tumorigenesis in a cell associated with the reproductive system of a mammal, by modulating the expression of a gene or the level and/or functional activity of an expression product of the gene, wherein the gene is selected from SOCS-1 or a gene belonging to the same regulatory or biosynthetic pathway as SOCS-1. The modulation preferably comprises contacting the cell with an agent that enhances the expression of the gene or the level and/or functional activity of the expression product, as for example described above. Such agents can be identified by any suitable method as for example disclosed in Section 4 infra.

Assays of a suitable nature for detecting, measuring or otherwise determining modulation of tumorigenesis (e.g., such as by detecting cell proliferation) are known to persons of skill in the art. For example, tumorigenesis-modulating agents could be tested for their ability to modulate cell proliferation. Typically, for cell proliferation, cell number is determined, directly, by microscopic or electronic enumeration, or indirectly, by the use of chromogenic dyes, incorporation of radioactive precursors or measurement of metabolic activity of cellular enzymes. An exemplary cell proliferation assay comprises culturing cells in the presence or absence of a test compound, and detecting cell proliferation by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65: 55-63). Cancer or tumour markers are known for a variety of cell or tissue types. Cells or tissues expressing cancer or tumour markers may be detected using monoclonal antibodies, polyclonal antisera or other antigen-binding molecules that are immuno-interactive with these tumour markers or by using nucleic acid analysis techniques, including, for example, detecting the level or presence of tumour marker-encoding polynucleotides. Alternatively, tumorigenesis can also be evaluated by conventional histological analysis.

4. Identification of Target Molecule Modulators

The invention also features a method of screening for an agent that modulates the level and/or functional activity of a target molecule comprising an expression product of a gene selected from a first gene encoding a SOCS-1 gene, or a second gene relating to the same regulatory or biosynthetic pathway as the first gene. The method comprises contacting a preparation comprising a first member selected from the expression product, or a biologically active fragment of the expression product, or a second member selected from a genetic sequence that regulates or encodes the expression product or a fragment of the genetic sequence, with a test agent, and detecting a change in the level and/or functional activity of the first member, or of an expression product relating to the second member.

Any suitable assay for detecting, measuring or otherwise determining modulation of differentiation and/or proliferation of mammary cells or modulation of mammopoiesis and lactogenesis is contemplated by the present invention. Assays of a suitable nature are known to persons of skill in the art and examples of these are described in Section 2 supra and the Examples infra.

Modulators contemplated by the present invention include agonists and antagonists of SOCS-1 gene expression or of SOCS-1 polypeptides. Antagonists of SOCS-1 gene expression include antisense molecules, ribozymes and co-suppression molecules, as for example described in Section 2. Agonists of SOCS-1 gene expression include molecules which increase promoter activity or which overcome any negative regulatory mechanism. Antagonists of SOCS-1 polypeptides include antibodies and inhibitor peptide fragments. Agonists of SOCS-1 polypeptides include SOCS-1 polynucleotides, or SOCS-1 polypeptides, or biologically active fragments thereof, or variants or derivatives, including mimetics, of these.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of a SOCS-1 polypeptide are particularly preferred. In this regard, small molecules are particularly preferred because such molecules are more readily absorbed after oral administration, have fewer potential antigenic determinants, and/or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening for modulatory agents according to the invention can be achieved by any suitable method. For example, the method may include contacting a cell comprising a polynucleotide corresponding to a SOCS-1 gene or a gene belonging to the same regulatory or biosynthetic pathway as the SOCS-1 gene, with an agent suspected of having the modulatory activity and screening for the modulation of the level and/or functional activity of a protein encoded by the polynucleotide, or the modulation of the level of an expression product encoded by the polynucleotide, or the modulation of the activity or expression of a downstream cellular target of the protein or of the expression product. Detecting such modulation can be achieved utilizing techniques including, but not restricted to, ELISA, cell-based ELISA, filter-binding ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced polynucleotide may be constitutively expressed—thereby providing a model useful in screening for agents which down-regulate expression of an encoded product of the sequence wherein the down regulation can be at the nucleic acid or protein level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g., a domain such as a protein molecule:interacting domain including, but not limited, to the SOCS box or the SH2 domain of a SOCS-1 polypeptide or variant or derivative thereof) or a portion that regulates expression of a product encoded by the polynucleotide (e.g., a promoter). For example, the promoter that is naturally associated with the polynucleotide may be introduced into the cell that is the subject of testing. In this regard, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (i.e., modulatory agents) which are capable of inducing or inhibiting the level and/or or functional activity of target molecules according to the invention. The assays may be performed in vitro using non-transformed cells, immortalised cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen-binding molecules disclosed herein), or increased or decreased levels of expression of a reporter gene (e.g., GFP, β-galactosidase or luciferase) operably linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in the level from an established baseline may be detected using any of the techniques described above and well known in the art. In particularly preferred embodiments, the cells are mammary cells, more preferably mammary epithelial cells and still more preferably mammary ductal epithelial cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modeling, and other routine procedures employed in rational drug design.

In another embodiment, a method of identifying agents that inhibit SOCS-1 activity is provided in which a purified preparation of an SOCS-1 protein is incubated in the presence and absence of a candidate agent under conditions in which the SOCS-1 is active, and the level of SOCS-1 activity is measured by a suitable assay. For example, a SOCS-1 inhibitor can be identified by measuring the ability of a candidate agent to decrease SOCS-1 activity in a cell (e.g., a mammary cell). In one embodiment of this method, a mammary ductal epithelial cell that is capable of expressing a SOCS-1 polynucleotide, is exposed to, or cultured in the presence and absence of, the candidate agent under conditions in which the SOCS-1 is active in the cell, and an activity relating to mammopoiesis and/or lactogenesis such as β-casein synthesis in, or differentiation of, the mammary ductal epithelial cell is detected. An agent tests positive if it potentiates or promotes this activity.

In yet another embodiment, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesized by any suitable technique. Such molecules may be conveniently prepared by a person skilled in the art using standard protocols as for example described in Sambrook, et al., (1989, supra) in particular Sections 16 and 17; Ausubel et al., (1994-1998, supra), in particular Chapters 10 and 16; and Coligan et al., (1995-1997, supra), in particular Chapters 1, 5 and 6. Alternatively, a target polypeptide according to the invention may be synthesized using solution synthesis or solid phase synthesis as described, for example, in Chapter 9 of Atherton and Shephard (supra) and in Roberge et al (1995, Science 269: 202).

To identify and isolate the peptide/solid phase support that interacts and forms a complex with a target molecule, preferably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labeling with enzymes, fluorescent dyes or colored or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-target polypeptide complex changes color, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labeled epitope specific antigen-binding molecule. Once isolated, the identity of the peptide attached to the solid phase support may be determined by peptide sequencing.

5. SOCS-1 Polypeptides

5.1 Wild-Type SOCS-1 Polypeptides

The invention encompasses the use of wild-type SOCS-1 polypeptides or biologically active fragments thereof for modulating differentiation and/or proliferation of mammary cells, for modulating tumorigenesis in a cell of the reproductive system or for identifying SOCS-1/SOCS-1 modulators. Exemplary human and murine amino acid sequences for SOCS-1 are set forth in the enclosed Sequence Listing infra and are summarized in TABLE A supra.

Exemplary biologically active fragments of SOCS-1, which are contemplated by the present invention, include but are not restricted to fragments comprising one or both of an SH2 domain and a SOCS box. The SH2 domain may comprise an amino acid sequence selected from SEQ ID NO: 5 or 6 but preferably comprises an amino acid sequence selected from SEQ ID NO: 7 or 8. Suitably, the SOCS box comprises an amino acid sequence selected from SEQ ID NO: 9, 10, 11 or 12.

5.2 SOCS-1 Variant Polypeptides

The invention contemplates the use of variants of wild-type SOCS-1 polypeptides or biologically active fragments thereof for modulating differentiation and/or proliferation of mammary cells, for modulating tumorigenesis in a cell of the reproductive system or for identifying SOCS-1/SOCS-1 modulators. Suitable methods of producing polypeptide variants include replacing at least one amino acid of a parent polypeptide comprising the sequence set forth in any one of SEQ ID NO: 2 or 4, or a biologically active fragment thereof, with a different amino acid to produce a modified polypeptide, and testing the modified polypeptide for an activity of the parent SOCS-1 polypeptide, including modulation of differentiation and/or proliferation of mammary epithelial cells, which indicates that the modified polypeptide is a polypeptide variant.

In another embodiment, a polypeptide variant is produced by replacing at least one amino acid of a parent polypeptide comprising the sequence set forth in any one of SEQ ID NO: 2 or 4, or a biologically active fragment thereof, with a different amino acid to produce a modified polypeptide, introducing the polypeptide or a polynucleotide from which the modified polypeptide can be translated into a cell having a deletion of, or disruption in, the SOCS-1 gene, and detecting an activity of the parent SOCS-1 polypeptide, including modulation of differentiation and/or proliferation of mammary cells, which indicates that the modified polypeptide is a polypeptide variant. Examples of assays that may be used in accordance with the present invention are described in Section 2.

In general, variants will be at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% homologous to a polypeptide as for example shown in any one of SEQ ID NO: 2 or 4 or a fragment thereof. Suitably, variants will have at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity to the sequence set forth in any one of SEQ ID NO: 2 or 4 or a fragment thereof.

Variant peptides or polypeptides, resulting from rational or established methods of mutagenesis or from combinatorial chemistries, for example, may comprise conservative amino acid substitutions. Exemplary conservative substitutions in a polypeptide or polypeptide fragment according to the invention may be made according to the following table:

TABLE B

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE B. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Asn) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a smaller side chain (e.g., Ala, Ser) or no side chain (e.g., Gly) is substituted for, or by, one having a bulky side chain (e.g., Phe or Trp).

5.3 Polypeptide Derivatives

The invention also extends to the use of SOCS-1 derivatives for modulating differentiation and/or proliferation of mammary cells and for identifying SOCS-1/SOCS-1 modulators. Such derivatives include amino acid deletions and/or additions to a polypeptide, fragment or variant of the invention, wherein the derivatives comprise an activity of a SOCS-1 polypeptide, including modulation of differentiation and/or proliferation of mammary cells. “Additions” of amino acids may include fusion of the polypeptides, fragments and polypeptide variants of the invention with other polypeptides or proteins. For example, it will be appreciated that the polypeptides, fragments or variants may be incorporated into larger polypeptides, and that such larger polypeptides may also be expected to modulate an activity as mentioned above.

The polypeptides, fragments or variants of the invention may be fused to a further protein, for example, which is not derived from the original host. The further protein may assist in the purification of the fusion protein. For instance, a polyhistidine tag or a maltose binding protein may be used in this respect as described in more detail below. Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS). The carboxyl group may be modified by carbodiimide activation via O-acylisourea formation followed by subsequent derivatisation, by way of example, to a corresponding amide. The guanidine group of arginine residues may be modified by formation of heterocyclic condensation products with reagents such as 2,3-butanedione, phenylglyoxal and glyoxal. Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulfides with other thiol compounds; reaction with maleimide, maleic anhydride or other substituted maleimide; carboxymethylation with iodoacetic acid or iodoacetamide; and carbamoylation with cyanate at alkaline pH. Tryptophan residues may be modified, for example, by alkylation of the indole ring with 2-hydroxy-5-nitrobenzyl bromide or sulphonyl halides or by oxidation with N-bromosuccinimide. Tyrosine residues may be modified by nitration with tetranitromethane to form a 3-nitrotyrosine derivative. The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in TABLE C.

TABLE C

| Non-conventional amino acid | Non-conventional amino acid |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Also contemplated is the use of crosslinkers, for example, to stabilize 3D conformations of the polypeptides, fragments or variants of the invention, using homo-bifunctional cross linkers such as bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety or carbodiimide. In addition, peptides can be conformationally constrained, for example, by introduction of double bonds between C_{α} and C_{β} atoms of amino acids, by incorporation of C_{α} and N_{α}-methylamino acids, and by formation of cyclic peptides or analogues by introducing covalent bonds such as forming an amide bond between the N and C termini between two side chains or between a side chain and the N or C terminus of the peptides or analogues. For example, reference may be made to: Marlowe (1993, Biorganic & Medicinal Chemistry Letters 3: 437-44) who describes peptide cyclisation on TFA resin using trimethylsilyl (TMSE) ester as an orthogonal protecting group; Pallin and Tam (1995, J. Chem. Soc. Chem. Comm. 2021-2022) who describe the cyclisation of unprotected peptides in aqueous solution by oxime formation; Algin et al (1994, Tetrahedron Letters 35: 9633-9636) who disclose solid-phase synthesis of head-to-tail cyclic peptides via lysine side-chain anchoring; Kates et al (1993, Tetrahedron Letters 34: 1549-1552) who describe the production of head-to-tail cyclic peptides by three-dimensional solid phase strategy; Tumelty et al (1994, J. Chem. Soc. Chem. Comm. 1067-1068) who describe the synthesis of cyclic peptides from an immobilized activated intermediate, wherein activation of the immobilized peptide is carried out with N-protecting group intact and subsequent removal leading to cyclisation; McMurray et al (1994, Peptide Research 7: 195-206) who disclose head-to-tail cyclisation of peptides attached to insoluble supports by means of the side chains of aspartic and glutamic acid; Hruby et al (1994, Reactive Polymers 22: 231-241) who teach an alternate method for cyclising peptides via solid supports; and Schmidt and Langer (1997, J. Peptide Res. 49: 67-73) who disclose a method for synthesizing cyclotetrapeptides and cyclopentapeptides. The foregoing methods may be used to produce conformationally constrained polypeptides that modulate differentiation and/or proliferation of mammary cells or that modulate tumorigenesis of cells that are associated with the reproductive system.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

6. Methods of Preparing a SOCS-1 Polypeptide

A SOCS-1 polypeptide, fragment or variant thereof may be prepared by any suitable procedure known to those of skill in the art. For example, SOCS-1 polypeptides, fragments or variants may be prepared by a procedure including the steps of (a) preparing a recombinant polynucleotide comprising a nucleotide sequence encoding a polypeptide comprising the sequence set forth in any one of SEQ ID NO: 2 or 4, or a biologically active fragment thereof, or variant or derivative of these, which nucleotide sequence is operably linked to regulatory elements; (b) introducing the recombinant polynucleotide into a suitable host cell; (c) culturing the host cell to express recombinant polypeptide from the recombinant polynucleotide; and (d) isolating the recombinant polypeptide. Preferred nucleotide sequences include, but are not limited to the sequences set forth in SEQ ID NO: 1 or 3.

The recombinant polynucleotide is preferably in the form of an expression vector that may be a self-replicating extra-chromosomal vector such as a plasmid, or a vector that integrates into a host genome. The regulatory elements will generally be appropriate for the host cell used for expression. Numerous types of appropriate expression vectors and suitable regulatory sequences are known in the art for a variety of host cells. Typically, the regulatory elements include, but are not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with the fusion partner. The main advantage of fusion partners is that they assist identification and/or purification of the fusion polypeptide. In order to express the fusion polypeptide, it is necessary to ligate a polynucleotide according to the invention into the expression vector so that the translational reading frames of the fusion partner and the polynucleotide coincide. Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter. Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localization of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be achieved by any suitable method including transfection, and transformation, the choice of which will be dependent on the host cell employed. Such methods are well known to those of skill in the art.

Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilized with a baculovirus expression system.

The recombinant protein may be conveniently prepared by a person skilled in the art using standard protocols as for example described in Sambrook, et al., 1989, in particular Sections 16 and 17; Ausubel et al., (1994-1998), in particular Chapters 10 and 16; and Coligan et al., (1995-1997), in particular Chapters 1, 5 and 6.

Alternatively, the SOCS-1 polypeptide, fragments, variants or derivatives may be synthesized using solution synthesis or solid phase synthesis as described, for example, in Chapter 9 of Atherton and Shephard (supra) and in Roberge et al (1995).

7. SOCS-1 Polynucleotides Variants

The present invention also envisions the use of SOCS-1 polynucleotide variants for modulating differentiation and/or proliferation of mammary cells and for identifying SOCS-1/SOCS-1 modulators. In general, SOCS-1 polynucleotide variants according to the invention comprise regions that show at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity over a reference polynucleotide sequence of identical size (“comparison window”) or when compared to an aligned sequence in which the alignment is performed by a computer homology program known in the art.

What constitutes suitable variants may be determined by conventional techniques. For example, a polynucleotide according to any one of SEQ ID NO: 1 or 3 can be mutated using random mutagenesis (e.g., transposon mutagenesis), oligonucleotide-mediated (or site-directed) mutagenesis, PCR mutagenesis and cassette mutagenesis as is known in the art.

Alternatively, suitable polynucleotide sequence variants of the invention may be prepared according to the following procedure: creating primers which are optionally degenerate wherein each comprises a portion of a reference polynucleotide encoding a reference polypeptide or fragment of the invention, preferably encoding the sequence set forth in SEQ ID NO: 2 or 4; obtaining a nucleic acid extract from an organism, which is preferably an animal, and more preferably a mammal; and using the primers to amplify, via nucleic acid amplification techniques, at least one amplification product from the nucleic acid extract, wherein the amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994); and Q-β replicase amplification as for example described by Tyagi et al., (1996).

Typically, polynucleotide variants that are substantially complementary to a reference polynucleotide are identified by blotting techniques that include a step whereby nucleic acids are immobilized on a matrix (preferably a synthetic membrane such as nitrocellulose), followed by a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridizing the membrane-bound DNA to a complementary nucleotide sequence labeled radioactively, enzymatically or fluorochromatically. In dot blotting and slot blotting, DNA samples are directly applied to a synthetic membrane prior to hybridization as above.

An alternative blotting step is used when identifying complementary polynucleotides in a cDNA or genomic DNA library, such as through the process of plaque or colony hybridization. A typical example of this procedure is described in Sambrook et al. (1989) Chapters 8-12.

Typically, the following general procedure can be used to determine hybridization conditions. Polynucleotides are blotted/transferred to a synthetic membrane, as described above. A reference polynucleotide such as a polynucleotide of the invention is labeled as described above, and the ability of this labeled polynucleotide to hybridize with an immobilized polynucleotide is analyzed.

A skilled artisan will recognize that a number of factors influence hybridization. The specific activity of radioactively labeled polynucleotide sequence should typically be greater than or equal to about 10⁸ dpm/mg to provide a detectable signal. A radiolabelled nucleotide sequence of specific activity 10⁸ to 10⁹ dpm/mg can detect approximately 0.5 μg of DNA. It is well known in the art that sufficient DNA must be immobilized on the membrane to permit detection. It is desirable to have excess immobilized DNA, usually 10 μg. Adding an inert polymer such as 10% (w/v) dextran sulphate (MW 500,000) or polyethylene glycol 6000 during hybridization can also increase the sensitivity of hybridization (see Ausubel supra at 2.10.10).

To achieve meaningful results from hybridization between a polynucleotide immobilized on a membrane and a labeled polynucleotide, a sufficient amount of the labeled polynucleotide must be hybridized to the immobilized polynucleotide following washing. Washing ensures that the labeled polynucleotide is hybridized only to the immobilized polynucleotide with a desired degree of complementarity to the labeled polynucleotide.

It will be understood that polynucleotide variants according to the invention will hybridize to a reference polynucleotide under at least low stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridize to a reference polynucleotide under at least medium stringency conditions. Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridize to a reference polynucleotide under high stringency conditions. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:
Tₘ=81.5+16.6(log₁₀ M)+0.41 (% G+C)−0.63 (% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In a preferred hybridization procedure, a membrane (e.g., a nitrocellulose membrane or a nylon membrane) containing immobilized DNA is hybridized overnight at 42° C. in a hybridization buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Methods for detecting a labeled polynucleotide hybridized to an immobilized polynucleotide are well known to practitioners in the art. Such methods include autoradiography, phosphorimaging, and chemiluminescent, fluorescent and colorimetric detection.

8. Enhancing Milk Yield in a Milk-Producing Animal

The invention encompasses a method of increasing milk yield in an animal, comprising administering to the animal a milk yield increasing effective amount of an agent that inhibits, abrogates or otherwise reduces expression of a SOCS-1 gene or of another gene belonging to the same regulatory or biosynthetic pathway as SOCS-1 or the level and/or functional activity of an expression product of the SOCS-1 gene or of the other gene to thereby potentiate or promote mammopoiesis and lactogenesis.

By “milk-producing animal” is intended, for the purpose of this invention, animals, preferably mammals, which produce milk in commercially feasible quantities, preferably cows, sheep, goats, buffalo and llamas among other and more preferably dairy cows of the genus Bos (bovid).

The milk yield-enhancing agents of the present invention can be administered orally or systemically. However, it is preferred that such agents are included as additives to feed which suitably comprises a basal diet for feeding an animal. When the feed additive of the present invention is added to feed, it may be formulated along with feed components at the time of feed formulation, or may be added to feed at the time of feeding to animals. There is no limitation on the method and time of addition to feed. The arbitrary basal diet for animals, which is used for preparing the feed according to the invention, is not particularly limited. Examples of raw materials, which may constitute a basal diet include, but are not limited to, grains such as corn, milo and wheat flour, brans such as defatted rice bran and wheat bran, animal substances such as fish meal and skim milk, vegetable oil cake such as soybean oil cake, and additives such as calcium carbonate, calcium phosphate, common salt, DL-methionine, choline chloride, manganese sulfate, dry iron sulfate, calcium iodate, copper sulfate, dry zinc sulfate and sodium saccharin. The basal diet can be prepared by blending together such raw materials. Carbohydrates are also suggested for addition to the basal diet as a source of energy and as a filler, preferably in amounts ranging from 1% to 35% by weight of the total feed. The basal diet may also comprise a small amount of fat, such as fish oil, as an additional source of energy preferably in amounts ranging from 1% to 5% by weight of the total feed. The formulation of the basal diet will vary depending on the animal to which the diet is fed. The feed may be in solid or liquid form. Supplemental vitamins, including additional ascorbic acid and Vitamin B₂, minerals and trace elements sufficient to meet the daily nutritional requirements of the animal may be included in the feed as well as other compounds such as anti-microbiles and antibiotics registered for use with animals for consumption.

The feed formulation may be processed by any means now known or later developed in the art, including extrusion or pelleting techniques. Extrusion generally adds an amount of air to the final product, such that the flakes prepared by extrusion usually float when added to water. Pelleting, on the other hand, generally provides a dense pellet that sinks upon addition to water. The desired form of the final feed formulation will depend upon the species and feeding habits of the fish being cultivated.

Other additives or adjuvants may also be added to the final processed feed as a coating where desired. For instance, it is known to coat processed feed pellets with an amount of oil or fat to enhance the water stability and cohesion of the pellets.

9. Therapeutic and Prophylactic Uses

In accordance with the present invention, it is proposed that agents (drugs) which directly or indirectly enhance the expression of SOCS-1 or the level and/or functional activity of an expression product (preferably SOCS-1) of SOCS-1, are useful as drugs for the modulation of tumorigenesis in cells which are associated with the reproductive system including, but not limited to, mammary cells and ovarian cells, and for the treatment and/or prophylaxis of cancer of a reproductive organ or a related cancer or condition. Such drugs can be administered to a patient either by themselves or in pharmaceutical compositions where they are mixed with a suitable pharmaceutically acceptable carrier.

Depending on the specific conditions being treated, the drugs may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the drugs of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The drugs can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of drug administered to a patient should be sufficient to achieve a beneficial response in the patient over time such as differentiation, preferably terminal differentiation, of mammary epithelial cells or a reduction in the proliferation or regression of a mammary carcinoma. The quantity of the drug(s) to be administered may depend on the subject to be treated inclusive of the age, sex, weight and general health condition thereof. In this regard, precise amounts of the drug(s) for administration will depend on the judgement of the practitioner. In determining the effective amount of the drug to be administered in the modulation of tumorigenesis, the physician may evaluate tissue levels of a SOCS-1 polypeptide, the level or tumor antigens or tumor growth or the differentiation of mammary epithelial cells. In any event, those of skill in the art may readily determine suitable dosages of the drugs of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more drugs as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the drugs of the invention may also include injecting or implanting controlled releasing devices designed specifically for this purpose or other forms of implants modified to act additionally in this fashion. Controlled release of an agent of the invention may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be achieved by using other polymer matrices, liposomes and/or microspheres.

The drugs of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the methods of the invention, the therapeutically effective dose can be estimated initially from cell culture assays. For example, a dose can be formulated in animal models to achieve a circulating concentration range, that includes the IC50 as determined in cell culture (e.g., the concentration of a test agent, which achieves a half-maximal inhibition in activity of a SOCS-1polypeptide). Such information can be used to more accurately determine useful doses in humans.

Toxicity and therapeutic efficacy of such drugs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975, in “The Pharmacological Basis of Therapeutics”, Ch. 1 p 1).

Dosage amount and interval may be adjusted individually to provide plasma levels of the active agent which are sufficient, for example, to maintain SOCS-1-inhibitory or enhancing effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is preferably testicular tissue, often in a depot or sustained release formulation. Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

The present invention also contemplates a method of “anti-sense therapy” of a mammal. Such a method utilizes an anti-sense therapy construct which includes an isolated polynucleotide comprising one or more selected portions of a SOCS-1 polynucleotide oriented 3′→5′ in a gene therapy vector, which provides one or more regulatory sequences that direct expression of the polynucleotide in the mammal. For example, epithelial cell-specific promoters, such as whey acidic protein (wap), can be used to target expression of a given antisense construct or other modulatory agent in ductal epithelial cells. Typically, gene therapy vectors are derived from viral DNA sequences such as adenovirus, adeno-associated viruses, herpes-simplex viruses and retroviruses. Suitable gene therapy vectors currently available to the skilled person may be found, for example, in Robbins et al. (1998, Pharmacol Ther. 80(1): 35-47).

In an alternate embodiment, a polynucleotide encoding a modulatory agent of the invention (e.g., a SOCS-1-expressing vector or a SOCS-1 antisense or ribozyme vector) may be used as a therapeutic or prophylactic composition in the form of a “naked DNA” composition as is known in the art. For example, an expression vector comprising the polynucleotide operably linked to a regulatory polynucleotide (e.g. a promoter, transcriptional terminator, enhancer etc) may be introduced into an animal, preferably a mammal, where it causes production of a modulatory agent in vivo, suitably in a reproductive tissue, preferably in mammary epithelium and more preferably in mammary ductal epithelium.

The step of introducing the expression vector into a mammary cell or tissue will differ depending on the intended use and species, and can involve one or more of non-viral and viral vectors, cationic liposomes, retroviruses, and adenoviruses such as, for example, described in Mulligan, R. C., (1993). Such methods can include, for example:
A. Local application of the expression vector by injection (Wolff et al., 1990), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of the protein.
B. General systemic delivery by injection of DNA, (Calabretta et al., 1993), or RNA, alone or in combination with liposomes (Zhu et al., 1993), viral capsids or nanoparticles (Bertling et al., 1991) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by the expression vector, or of cells responsive to the protein.
C. Injection or implantation or delivery by any means, of cells that have been modified ex vivo by transfection (for example, in the presence of calcium phosphate: Chen et al., 1987, or of cationic lipids and polyamines: Rose et al., 1991), infection, injection, electroporation (Shigekawa et al., 1988) or any other way so as to increase the expression of the polynucleotide in those cells. The modification can be mediated by plasmid, bacteriophage, cosmid, viral (such as adenoviral or retroviral; Mulligan, 1993; Miller, 1992; Salmons et al., 1993) or other vectors, or other agents of modification such as liposomes (Zhu et al., 1993), viral capsids or nanoparticles (Bertling et al., 1991), or any other mediator of modification. The use of cells as a delivery vehicle for genes or gene products has been described by Barr et al., 1991 and by Dhawan et al., 1991. Treated cells can be delivered in combination with any nutrient, growth factor, matrix or other agent that will promote their survival in the treated subject.

However, it will be understood that all modes of delivery of nucleic acid compositions are contemplated by the present invention. Delivery of these compositions to mammary cells or tissues of an animal may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of CURRENT PROTOCOLS IN MOLECULAR BIOLOGY (Eds. Ausubel et al.; John Wiley & Sons Inc., 1997 Edition) or on the Internet site DNAvaccine.com.

In one embodiment of a prophylactic method of the present invention, the epithelium of a mammary gland is treated prophylactically for cancer so as to inhibit the formation of cancer of epithelial origin. The method comprises contacting (e.g., by ductal cannulation) the epithelium, preferably the ductal epithelium, of the mammary gland with an agent which directly or indirectly inhibits or enhances the expression of SOCS-1 or the level and/or functional activity of an expression product (preferably SOCS-1-1) of SOCS-1.

The above-described prophylactic method of treating a mammary gland is particularly useful in treating a mammary gland in a mammal at risk for developing breast cancer. The mammary gland can be characterized as one that has never had a tumor, one that had a tumor previously but the tumor is no longer detectable due to other prior therapeutic treatment, or one that has an incipient or occult tumor, preneoplasia or ductal hyperplasia. Normally, hyperplasias and incipient and occult tumors are not detectable by means of physical examination or radiography. Accordingly, the prophylactic method will find use in cases where there is reason to take some prophylactic measures, such as when there are known inherited factors predisposing to cancers, where there are suspicious lesions present in a breast with the potential for developing into a malignancy, where there has been exposure to carcinogenic agents in the environment, where age predisposes to a cancer, where cancer of another gland, e.g., the mammary gland of the contralateral breast, suggests a propensity for developing cancer, or where there is a fear or suspicion of metastasis.

The ductal epithelium is preferably contacted with the agent by introduction of the agent through the central canal or duct of the exocrine ductal epithelium, such as by ductal cannulation. However, in the case of the mammary gland, for example, there are 6-9 major ducts that emanate from the nipple and serially branch into other ducts, terminating in lobuloalveolar structures (Russo et al. 1990, Laboratory Investigation 62: 244-278). Accordingly, in some circumstances, such as those in which even more localized treatment is necessary or desired, for example, by the choice of anti-cancer agent, it may be preferable to contact the ductal epithelium of the mammary gland through one of the other ducts or through a lobuloalveolar structure as opposed to the central canal or duct. In this regard, ductal cannulation enables intratumoral injection.

In order that the invention may be readily understood and put into practical effect, particular preferred embodiments will now be described by way of the following non-limiting examples.

In order that the invention may be readily understood and put into practical effect, particular preferred embodiments will now be described by way of the following non-limiting example.

### EXAMPLES

### Example 1

SOCS-1 is Expressed in the Developing Mammary Gland

In situ hybridization revealed that SOCS-1 RNA is highly expressed in the ductal epithelium and lobuloalveolar units of the developing mammary gland and is apparent, at lower levels, in the surrounding stroma. SOCS-1 RNA appeared to be more abundant in the developing lobuloalveolar units of mammary glands during pregnancy. RT-PCR analysis of mammary tissue from different stages of development confirmed that the level of SOCS-1 RNA was higher (at least 5-fold) in glands from pregnant females relative to those from lactating or involuting glands (data not shown).

Methods:

In Situ Hybridisation

Full-length mouse SOCS-1 cDNA (Starr et al. 1997) was cloned into Bluescript SKII (Stratagene). Antisense and sense riboprobes were generated using T3 or T7 RNA polymerase (Promega) with digoxigenin-UTP (Roche). Standard in situ hybridisations were performed as described (Wilkinson 1992).

### Example 2

Overexpression of SOCS Genes Inhibits β-Casein Synthesis in Mammary Epithelial Cells

To examine the role of SOCS-1 genes in mammary differentiation, we utilized the mammary epithelial line, SCp2, which displays the essential features of mammary differentiation in the presence of extracellular matrix (ECM) and a lactogenic stimulus (Desprez et al. 1993). Differentiation of these cells is accompanied by the production of milk proteins, such as β-casein, which we have used here as a molecular marker. Linearized expression vectors containing either SOCS-1, SOCS-2, SOCS-3 or CIS carrying an N-terminal Flag or GFP tag, plus a puromycin resistance cassette, were introduced into SCp2 cells and pools of stable transfectants assayed for their ability to undergo differentiation. For the latter assay, transfectants were plated on ECM in the presence or absence of a lactogenic stimulus.

All four SOCS genes were found to profoundly inhibit β-casein synthesis by 10- to 50-fold, while transfectants expressing vector alone were indistinguishable from the parental cells. Expression of the Flag-tagged SOCS-1 and SOCS-2 transgenes was readily detectable in SCp2 cells while Flag-SOCS-3 was undetectable, probably accounting for the weaker inhibition observed. However, expression of a GFP-tagged SOCS-3 transgene proved to be more stable in these cells and, accordingly, was more effective in blocking β-casein mRNA synthesis. Thus, SOCS-1 to -3 and CIS can all act as negative regulators of the endogenous prolactin signaling pathway in SCp2 cells, despite these genes exhibiting widely different levels of expression in mammary epithelium (data not shown).

Methods:

SCp2 Cell Differentiation Assay

SCp2 mammary epithelial cells (Desprez et al. 1993) were passaged in DMEM-F12 media containing DMEM-HAM, 10% FCS, and insulin 5 μg/ml (Sigma). Full-length cDNAs corresponding to SOCS-1 to -3 (Hilton et al. 1998) and CIS (Yasukawa et al. 1999), all carrying an N-terminal FLAG-epitope tag, were cloned into the pEF1α-puro mammalian expression vector (Huang et al. 1997). Protein expression was confirmed by transient transfection of 293T cells. Linearised expression vectors (10 μg) were introduced into SCp2 cells using Superfect (Qiagen) and selected in puromycin for 8 days. Pools of stable transfectants were then used in the differentiation assay, essentially as described (Desprez et al. 1993).

RNA Analysis and RT-PCR

RNA was isolated from SCp2 cells on ECM using RNAzol (Tel-Test); cDNA synthesis and PCR were performed as described (Weiss et al. 1994), using primers for β-casein and HPRT (Weiss et al. 1994). Sequences of the β-casein primers were: forward 5′-ATGAAGGTCTTCATCCTCGCCTGCC-3′, [SEQ ID NO: 13] reverse 5′-GCTGGACCAGAGACTGAGGAAGGTGC-3′ [SEQ ID NO: 14]. Northern analysis of total RNA was performed as described (Weiss et al. 1994).

Immunoprecipitation and Western Analysis

Whole cell lysates were generated from stably transfected SCp2 pools by lysing cells in KALB lysis buffer containing protease inhibitors (Complete Cocktail, Roche). Proteins were immunoprecipitated with anti-Flag M2 (Sigma) or rabbit antiserum raised against full-length SOCS-3, and protein G Sepharose (Pharmacia), and separated by SDS-PAGE (Novex). After transfer, filters were blocked and incubated with mouse anti-SOCS-1, rat anti-Flag or mouse anti-SOCS-3 (N-terminus) monoclonal antibodies. Antibody binding was visualised with peroxidase-conjugated anti-mouse (Amersham) or anti-rat antibody (Jackson Immunoresearch Laboratories) using the ECL system (Amersham).

Mouse mammary gland lysates were prepared in 150 mM NaCl, 5 mM EDTA, 50 mM Tris-Cl pH 7.5, 0.1% NP-40, and 0.1% deoxycholate containing protease inhibitors. After protein fractionation and transfer, filters were blocked with 50 mM sodium phosphate pH 7.0, 50 mM NaCl, 0.05% Tween 20, and incubated with one of the following primary antibodies: rabbit polyclonal antiserum raised against mouse milk-specific proteins (Accurate Chemical & Scientific Corporation), anti-phospho-Stat5a/b or anti-Stat5a monoclonal antibody (Upstate Biotech), anti-ERK1/2 (p44/42 MAPK) or anti-phospho ERK1/2 monoclonal antibody (New England Biolabs), anti-α-tubulin monoclonal antibody (Sigma). For milk protein detection, 400 ng of protein was loaded per lane, while other blots were performed using 20 μg protein per lane.

### Example 3

SOCS-1 Deficiency Accelerates Lobuloalveolar Development

Since targeted deletion of the IFNγ gene rescues SOCS-1^{-/-} mice from death at two weeks of age (Alexander et al. 1999; Marine et al. 1999b), these double knock-out mice could be used to study the effect of SOCS-1 deficiency on mammopoiesis by comparison with mice lacking IFNγ alone. SOCS-1^{-/-}/IFNγ^{-/-} mice were crossed to generate females for developmental analysis while SOCS-1^{+/+}/IFNγ^{-/-} mice were bred to generate control IFNγ^{-/-} females. Between 3 and 7 age-matched female mice of each genotype were analyzed at different stages of development. Importantly, loss of IFNγ had no discernible effect on mammary development and these mice appeared identical to wild type mice at all stages of development. No overt differences were found between mammary glands from SOCS-1^{-/-}/IFNγ^{-/-} females versus those from IFNγ^{-/-} or wild type mice at 4, 6, 9, 12, 15 and 18 weeks (data not shown).

SOCS-1 deficiency led to increased development of the lobuloalveolar units during pregnancy, as revealed by wholemount analysis and histological sectioning. There was a markedly higher density of lobuloalveolar units in mammary glands from SOCS-1^{-/-}/IFNγ^{-/-} mice, apparent from day 16 of pregnancy, relative to those from control mice. By day 18 of pregnancy, these units had substantially penetrated the mammary fat pad and displayed dilated lumens. Although development was more advanced at day 1 of lactation in the double knockout females, there was no difference by day 5. The rate of proliferation appeared to be similar for SOCS^{-/-}/IFNγ^{-/-} and IFNγ^{-/-} mammary epithelium, based on BrdU staining at days 13 and 16 of pregnancy (data not shown).

Methods:

Derivation and Maintenance of Mice

The derivation of SOCS-1^{-/-} and IFNγ^{-/-} mice has been described previously (Alexander et al. 1999). SOCS-1^{-/-} mice were originally maintained on a hybrid 129/Sv and C57BL/6 (SVB6) genetic background, while IFNγ^{-/-} mice were on an inbred C57BL/6 background. SOCS-1^{-/-}/IFNγ^{-/-}, SOCS-1^{+/+}/IFNγ^{-/-} and SOCS-1^{+/+}/IFNγ^{+/+} mice were generated by crosses and then propagated as intercrosses. Prolactin Receptor PRLR^{+/-} (129Ola/129SvPas background) mice were mated with SVB6 or 129Sv SOCS-1^{+/-} animals to generate pups heterozygous for both alleles on two different backgrounds. Mice were genotyped by Southern blot analysis (SOCS-1) and PCR of genomic tail DNA (PRLR) (Ormandy et al. 1997; Alexander et al. 1999). Mice were routinely housed in conventional facilities at the Walter & Eliza Hall Institute for Medical Research.

Adult female mice were mated and pregnancy scored by the observation of a vaginal plug and confirmed by examination of embryos when mammary glands were collected. Following parturition, litters with at least six pups were maintained. Pups were removed after 7-10 days to initiate involution.

Histology, Mammary Gland Wholemounts and Transplants

For histological examination, tissues were fixed in 10% (v/v) formalin in phosphate-buffered saline (PBS), embedded in paraffin and sections (1.5 μm) prepared and stained with hematoxylin and eosin (H&E staining). For wholemount examination, tissues were fixed in Carnoy's solution and stained with hematoxylin or carmine alum. Epithelial transplants into cleared mammary fat pads were carried out as described (Brisken et al. 1999).

### Example 4

Increased Milk Production in the Absence of SOCS-1

The dilated acini evident in mammary glands from day 18 pregnant and day 1 lactating SOCS-1^{-/-}/IFNγ^{-/-} mice suggested increased production and secretion of milk. Western analysis of whole-cell extracts from double knockout and age-matched control mice using anti-mouse milk antisera confirmed that there were significantly higher levels of milk proteins in the mammary gland in the absence of SOCS-1. Milk protein expression, including the expression of WAP (14 kD), α-casein (46 kD) and β-casein (30 kD), was markedly upregulated in three sets of mice at day 18 of pregnancy. Milk protein levels were elevated from day 16 of pregnancy through to day 1 of lactation in SOCS-1^{-/-}/IFNγ^{-/-} mammary glands relative to those from control mice, with the maximal difference occurring at day 18 of pregnancy.

Since Stat5 is an important transcriptional effector in the prolactin pathway (Liu et al. 1997; Teglund et al. 1998) and is known to directly regulate expression of milk protein genes, we examined whether phosphorylation of Stat5 was elevated in SOCS-1^{-/-}/IFNγ^{-/-} mice. Higher levels of phosphorylated Stat5 were found in mammary glands at day 1 of lactation relative to controls, although there was no apparent difference during pregnancy. Furthermore, there was no change in Stat5 DNA-binding activity during pregnancy (data not shown). Interestingly, substantially less MAP kinase activity (phospho-Erk1 and phospho-Erk2) was found in SOCS-1^{-/-}/IFNγ^{-/-} mammary glands at day 18 of pregnancy and day 1 of lactation, relative to control mammary tissue. The level of total Erk1/2 remained the same, indicating that MAP kinase activity was reduced. It is not known whether SOCS-1 directly influences MAP kinase activity but the diminished levels most likely reflect the terminal differentiated state of the epithelium.

### Example 5

Deletion of One SOCS-1 Allele Rescues the Lactogenic Defect Exhibited by Prolactin Receptor Heterozygous Females

Young PRLR^{+/-} females fail to lactate after their first pregnancy due to impaired lactogenesis but can lactate after subsequent pregnancies (Ormandy et al. 1997). Thus a single functional allele of PRLR is insufficient to drive the final rounds of epithelial differentiation and lactogenesis. This mammary defect varies in its penetrance, dependent on strain background.

To determine whether the lactogenic defect in PRLR mice was epithelial specific, we used epithelial explants from PRLR^{+/-} or PRLR^{+/+} mice transplanted into the cleared mammary fat pads of Rag1^{-/-} recipients. Reconstitution of wild type stroma with PRLR^{+/-} epithelium failed to rescue lobuloalveolar development during pregnancy, providing direct evidence that the defect lies in the epithelium. Moreover, recombination experiments using PRLR^{-/-} epithelium or stroma revealed that PRLR is required in the mammary epithelium but not in the stroma for normal development (MJN and CJO, data not shown).

To examine whether a reduction in the level of SOCS-1 might rescue signal transduction along the prolactin pathway, we generated females that were heterozygous for both PRLR and SOCS-1 and compared these to either SOCS-1^{+/-}, PRLR^{+/-} or wild type littermates. We found that six out of six double heterozygous females were capable of lactation after their first pregnancy, whereas four out of six PRLR^{+/-} females exhibited reduced lactation. Wholemount and histological analysis of glands from the rescued mice revealed normal morphology of the lobuloalveolar structures in PRLR^{+/-}/SOCS-1^{+/-} mice at day 2 postpartum but dramatically reduced development in four PRLR^{+/-} females. The rescue of lobuloalveolar development was also achieved in PRLR^{+/-}/SOCS-1^{+/-} mice on a different SOCS-1 (129Sv) background. Expression of WAP and β-casein milk protein genes in PRLR^{+/-}/SOCS-1^{+/-} mammary glands was restored to that seen in wild type glands, in contrast to the lower levels evident in PRLR^{+/-} mice.

From the foregoing, the present inventors have provided evidence that SOCS-1 is a negative regulator of prolactin signaling in vivo using two different sets of targeted mice. The precocious lobuloalveolar development that occurs in SOCS-1/IFNγ-deficient females but not those lacking IFNγ alone is compatible with SOCS-1 acting as an inhibitor. PRLR^{+/-} mice exhibit a specific defect in mammary differentiation. Whilst the architecture of the lobuloalveolar units is normal in these mice, the alveoli fail to dilate with milk, due to lack of terminal differentiation. Rescue of the lactogenic defect in PRLR^{+/-} females by removal of a single SOCS-1 allele demonstrates that SOCS-1 is indeed affecting prolactin signal transduction. The threshold level of PRLR required for terminal epithelial differentiation and lactogenesis is presumably restored by reducing the level of a negative regulator of this pathway.

Mammary gland development is not defective in mice lacking SOCS-1 and IFNγ but is accelerated. Expansion and maturation of the lobuloalveolar system are achieved earlier in the absence of SOCS-1. The morphological effects of SOCS-1 deficiency are first seen around day 16 of pregnancy but are no longer evident by day 5 of lactation. Since the lactogenic defect in PRLR^{+/-} mice is epithelial-specific, rescue of this phenotype by diminution of SOCS-1 indicates that SOCS-1 is acting cell autonomously within the epithelium. This is consistent with expression of SOCS-1 in the ductal epithelium and its prolactin inducibility in breast epithelial cells (Pezet et al. 1999; data not shown). It is plausible that SOCS-1 deficiency may have additional effects via the stroma, which could be addressed by reciprocal transplantation studies. It is notable that serum prolactin levels in adult SOCS-1^{-/-}/IFNγ^{-/-}, IFNγ^{-/-} and wild-type mice were within the normal range (data not shown). This finding further supports a direct role for SOCS-1 in the mammary gland.

It is believed that SOCS-1 may play a negative regulatory role in the induction of lactation after parturition. Lactation is a complex process that is determined in part by a postpartum decrease in progesterone and increase in serum prolactin levels (Wilde and Hurley 1996). The inhibition of lactation that is normally relieved at parturition is lost early in the SOCS-1^{-/-}/IFNγ^{-/-} mice, resulting in precocious lactation.

The signal transduction pathways regulated by SOCS-1 in the mammary gland remain to be defined. SOCS-1 can regulate cytokine signal transduction through direct inhibition of the Jak family of protein tyrosine kinases (Endo et al. 1997; Naka et al. 1997) and Stat5 is a direct target of Jak2. Consistent with these findings, an increase in phosphorylated Stat5 was observed at day 1 of lactation in SOCS-1^{-/-}/IFNγ^{-/-} glands relative to control glands. However, no increase was apparent during pregnancy when the phenotype was first manifest. These results suggest that the Stat5-response is prolonged but not amplified in the mammary glands of IFNγ^{-/-}/SOCS-1^{-/-} mice, as has been observed for Stat1 in hepatocytes from these mice. Alternatively, another SOCS-1-regulated pathway may also contribute to prolactin signaling. The observation that Stat5a-null mice are capable of milk protein synthesis invokes additional pathways in signaling by prolactin. The identification of such pathways should provide insight into the molecular basis of SOCS-1 inhibition in the mammary gland.

### Example 6

Analysis of Carcinomas

SOCS-1^{-/-}/IFNγ^{-/-} females and IFNγ^{-/-} control animals are monitored for the development of mammary tumours over the course of 18 months. To promote ductal proliferation in their mammary glands, a separate cohort of animals undergoes 3-4 rounds of pregnancy. Cohorts of ten animals are analysed at 6, 12 and 18 months of age. Wholemount analysis of unilateral 3^{rd} and 4^{th} mammary glands is carried out, and portions of contralateral glands are collected for histologic analysis, as well as for protein and RNA analysis. Histological assessment can then be used to focus on the presence of ductal or lobuloalveolar hyperplasia for in situ cancer, adenoma formation (including lactogenic adenomata or hyperplastic alveolar nodules) and frank adenoacarcinoma development. A complete autopsy is then performed on these mice. Mice that develop an interval malignancy are autopsied and mammary tissue analysed histologically. If a high incidence of mammary tumours is observed, premalignant phase mice are examined prior to frank tumour appearance. Tumours that are identified at autopsy undergo further analysis. Specifically, tumours are diced in sterile medium for serial transplantation in vivo and for culture in vitro, as well as portions snap frozen for protein and RNA analysis. The rate of proliferation is determined by BrdU analysis involving routine injection of mice with BrdU 45-60 minutes prior to collection of tissues. To determine the molecular mechanism by which mammary tumours arise, the phosphorylation status of Stat transcription factors and transducers can be determined in other pathways, including MAPK.

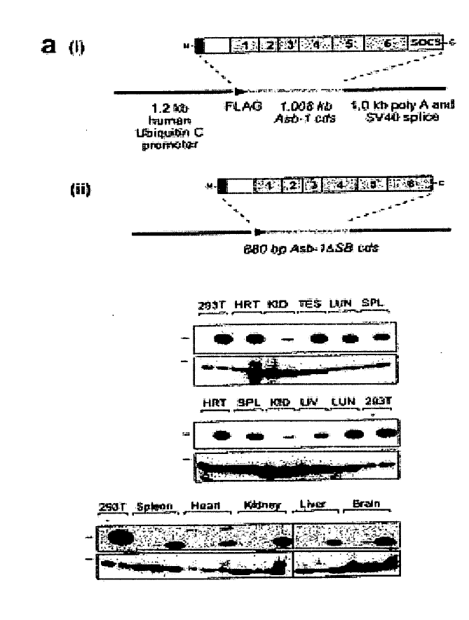

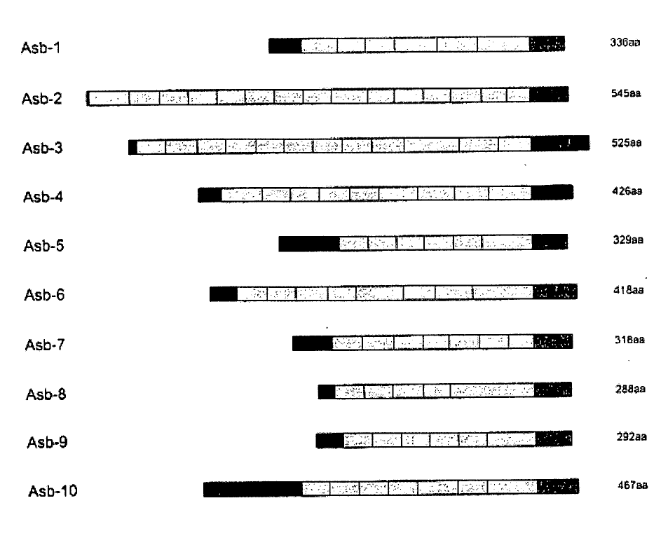

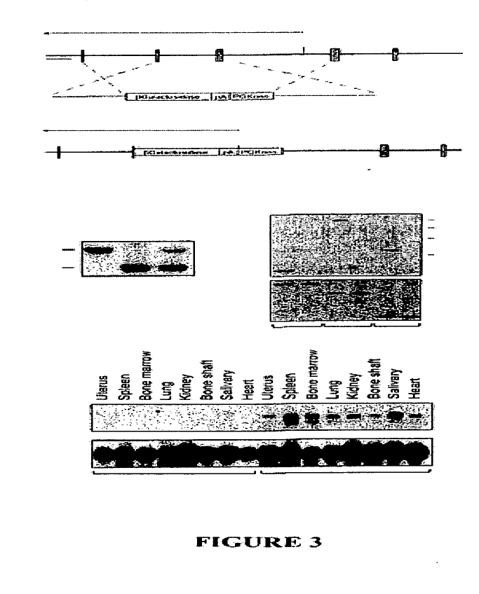

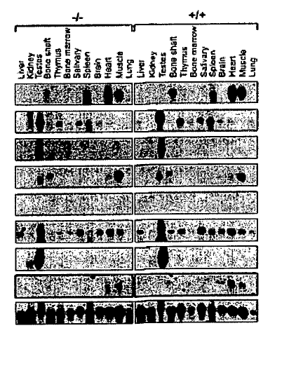

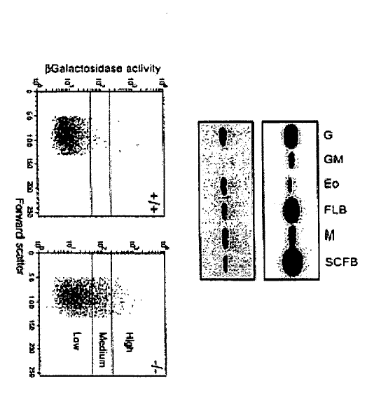

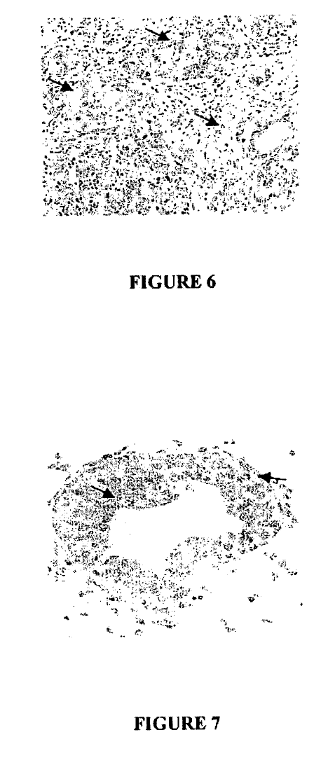

To date, the present inventors have analysed five female SOCS-1^{-/-}/IFNγ^{-/-} animals at 1 year of age. One of these was found to have an adnexal mass at autopsy, abutting the uterus and causing left ureteric obstruction and hydronephrosis. Histologic analysis revealed the mass to be a high grade adenocarcinoma, consistent with derivation in ovary (FIG. 1). Since ovarian cancer is a rare occurrence in wild-type animals and is not reported in IFNγ knockout animals, it is conceivable that SOCS-1 deficiency contributed to the tumour onset in this animal. Only one SOCS-1^{-/-}/IFNγ^{-/-} female has been analysed at 18 months of age. While wholemount analysis of the mammary gland was unremarkable, H&E sections revealed several foci of atypical ductal hyperplasia, containing apoptotic figures, representing a pre-malignant phenotype (FIG. 2). These changes were not present in a SOCS-1^{+/+}/IFNγ^{-/-} control.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Alexander, W. S., R. Starr, J. E. Fenner, C. L. Scott, E. Handman, N. S. Sprigg, J. E. Corbin, A. L. Cornish, R. Darwiche, C. M. Owczarek, T. W. Kay, N. A. Nicola, P. J. Hertzog, D. Metcalf, and D. J. Hilton. 1999. SOCS-1 is a critical inhibitor of interferon gamma signaling and prevents the potentially fatal neonatal actions of this cytokine. Cell 98: 597-608.
Ali, S., Z. Chen, J. J. Lebrun, W. Vogel, A. Kharitonenkov, P. A. Kelly, and A. Ullrich. 1996. PTP1D is a positive regulator of the prolactin signal leading to beta-casein promoter activation. EMBO J 15: 135-142.
Bole-Feysot, C., V. Goffin, M. Edery, N. Binart, and P. A. Kelly. 1998. Prolactin (PRL) and its receptor: actions, signal transduction pathways and phenotypes observed in PRL receptor knockout mice. Endocr Rev 19: 225-268.
Brisken, C., S. Kaur, T. E. Chavarria, N. Binart, R. L. Sutherland, R. A. Weinberg, P. A. Kelly, and C. J. Ormandy. 1999. Prolactin controls mammary gland development via direct and indirect mechanisms. Dev Biol 210: 96-106.
Desprez, P.-Y., C. Roskelley, J. Campisi, and M. J. Bissell. 1993. Isolation of functional cell lines from a mouse mammary epithelial cell strain: the importance of basement membrane and cell-cell interaction. Mol Cell Diff 1: 99-110.
Endo, T. A., M. Masuhara, M. Yokouchi, R. Suzuki, H. Sakamoto, K. Mitsui, A. Matsumoto, S. Tanimura, M. Ohtsubo, H. Misawa, T. Miyazaki, N. Leonor, T. Taniguchi, T. Fujita, Y. Kanakura, S. Komiya, and A. Yoshimura. 1997. A new protein containing an SH2 domain that inhibits JAK kinases. Nature 387: 921-924.
Helman, D., Y. Sandowski, Y. Cohen, A. Matsumoto, A. Yoshimura, S. Merchav, and A. Gertler. 1998. Cytokine-inducible SH2 protein (CIS3) and JAK2 binding protein (JAB) abolish prolactin receptor-mediated STAT5 signaling. FEBS Lett 441: 287-291.
Hennighausen, L., G. W. Robinson, K. U. Wagner, and W. Liu. 1997. Prolactin signaling in mammary gland development. J Biol Chem 272: 7567-7569.
Hilton, D. J., R. T. Richardson, W. S. Alexander, E. M. Viney, T. A. Willson, N. S. Sprigg, R. Starr, S. E. Nicholson, D. Metcalf, and N. A. Nicola. 1998. Twenty proteins containing a C-terminal SOCS box form five structural classes. Proc Natl Acad Sci 95: 114-119.
Horseman, N. D., W. Zhao, E. Montecino-Rodriguez, M. Tanaka, K. Nakashima, S. J. Engle, F. Smith, E. Markoff, and K. Dorshkind. 1997. Defective mammopoiesis, but normal hematopoiesis, in mice with a targeted disruption of the prolactin gene. EMBO J 16: 6926-6935.
Huang, D. C., S. Cory, and A. Strasser. 1997. Bcl-2, Bcl-XL and adenovirus protein E1B19kD are functionally equivalent in their ability to inhibit cell death. Oncogene 14: 405-414.
Krebs, D. L. and D. J. Hilton. 2000. SOCS: physiological suppressors of cytokine signaling. J Cell Sci 113: 2813-2819.
Liu, X., G. W. Robinson, K. U. Wagner, L. Garrett, A. Wynshaw-Boris, and L. Hennighausen. 1997. Stat5a is mandatory for adult mammary gland development and lactogenesis. Genes Dev 11: 179-186.
Marine, J. C., D. J. Topham, C. McKay, D. Wang, E. Parganas, D. Stravopodis, A. Yoshimura, and J. N. Ihle. 1999a. SOCS-1 deficiency causes a lymphocyte-dependent perinatal lethality. Cell 98: 609-616.
Marine, J. C., C. McKay, D. Wang, D. J. Topham, E. Parganas, H. Nakajima, H. Pendeville, H. Yasukawa, A. Sasaki, A. Yoshimura, and J. N. Ihle. 1999b. SOCS3 is essential in the regulation of fetal liver erythropoiesis. Cell 98: 617-627.
Matsumoto, A., Y. Seki, M. Kubo, S. Ohtsuka, A. Suzuki, I. Hayashi, K. Tsuji, T. Nakahata, M. Okabe, S. Yamada, and A. Yoshimura. 1999. Suppression of STAT5 functions in liver, mammary glands, and T cells in cytokine-inducible SH2-containing protein 1 transgenic mice. Mol Cell Biol 19: 6396-6407.
Naka, T., M. Narazaki, M. Hirata, T. Matsumoto, S. Minamoto, A. Aono, N. Nishimoto, T. Kajita, T. Taga, K. Yoshizaki, S. Akira, and T. Kishimoto. 1997. Structure and function of a new STAT-induced STAT inhibitor. Nature 387: 924-929.
Naka, T., T. Matsumoto, M. Narazaki, M. Fujimoto, Y. Morita, Y. Ohsawa, H. Saito, T. Nagasawa, Y. Uchiyama, and T. Kishimoto. 1998. Accelerated apoptosis of lymphocytes by augmented induction of Bax in SSI-1 (STAT-induced STAT inhibitor-1) deficient mice. Proc Natl Acad Sci 95: 15577-15582.
Ormandy, C. J., A. Camus, J. Barra, D. Damotte, B. Lucas, H. Buteau, M. Edery, N. Brousse, C. Babinet, N. Binart, and P. A. Kelly. 1997. Null mutation of the prolactin receptor gene produces multiple reproductive defects in the mouse. Genes Dev 11: 167-178.
Pezet, A., H. Favre, P. A. Kelly, and M. Edery. 1999. Inhibition and restoration of prolactin signal transduction by suppressors of cytokine signaling. J Biol Chem 274: 24497-24502.
Starr, R., T. A. Willson, E. M. Viney, L. J. Murray, J. R. Rayner, B. J. Jenkins, T. J. Gonda, W. S. Alexander, D. Metcalf, N. A. Nicola, and D. J. Hilton. 1997. A family of cytokine-inducible inhibitors of signalling. Nature 387: 917-921.
Starr, R., D. Metcalf, A. G. Elefanty, M. Brysha, T. A. Willson, N. A. Nicola, D. J. Hilton, and W. S. Alexander. 1998. Liver degeneration and lymphoid deficiencies in mice lacking suppressor of cytokine signaling-1. Proc Natl Acad Sci 95: 14395-14399.
Teglund, S., C. McKay, E. Schuetz, J. M. van Deursen, D. Stravopodis, D. Wang, M. Brown, S. Bodner, G. Grosveld, and J. N. Ihle. 1998. Stat5a and Stat5b proteins have essential and nonessential, or redundant, roles in cytokine responses. Cell 93: 841-850.
Tomic, S., N. Chughtai, and S. Ali. 1999. SOCS-1, -2, -3: selective targets and functions downstream of the prolactin receptor. Mol Cell Endocrinol 158: 45-54.
Vonderhaar, B. K. 1987. Prolactin: Transport, function, and receptors in mammary gland development and differentiation. In The mammary gland (ed. M. C. Neville and C. W. Daniel), pp. 383-438. Plenum Press, New York.
Watson, C. J. and T. G. Burdon. 1996. Prolactin signal transduction mechanisms in the mammary gland: the role of the Jak/Stat pathway. Rev Reprod 1:1-5.
Weiss, M. J., G. Keller, and S. H. Orkin. 1994. Novel insights into erythroid development revealed through in vitro differentiation of GATA-1 embryonic stem cells. Genes Dev 8: 1184-1197.
Wilde, C. J. and W. L. Hurley. 1996. Animal models for the study of milk secretion. J Mammary Gland Biol Neoplasia 1: 123-134.
Wilkinson, D. 1992. In Situ Hybridisation. IRL, New York.
Yasukawa, H., H. Misawa, H. Sakamoto, M. Masuhara, A. Sasaki, T. Wakioka, S. Ohtsuka, T. Imaizumi, T. Matsuda, J. N. Ihle, and A. Yoshimura. 1999. The JAK-binding protein JAB inhibits Janus tyrosine kinase activity through binding in the activation loop. EMBO J 18: 1309-1320.
Yoshimura, A. 1998. The CIS/JAB family: novel negative regulators of JAK signaling pathways. Leukemia 12: 1851-1857.

What is claimed is:

1. A method for modulating the differentiation and/or proliferation of a mammary cell, comprising modulating in the mammary cell the expression of a gene or the level and/or functional activity of an expression product of the gene, wherein the gene is selected from SOCS-1 or a gene belonging to the same regulatory or biosynthetic pathway as SOCS-1.

2. A method for modulating the differentiation and/or proliferation of a mammary cell, comprising modulating in the mammary cell the expression of SOCS-1 or the level and/or functional activity of an expression product of SOCS-1.

3. The method of claim 1 or claim 2, wherein the expression of SOCS-1 or the level and/or functional activity of the SOCS-1 expression product is reduced or abrogated.

4. The method of claim 1 or claim 2, wherein the expression of SOCS-1 or the level and/or functional activity of the SOCS-1 expression product is reduced or abrogated using a construct from which an antisense molecule is producible that is substantially complementary to at least a portion of SOCS-1.

5. The method of claim 1 or claim 2, wherein the expression of SOCS-1 or the level and/or functional activity of the SOCS-1 expression product is reduced or abrogated using a construct from which a ribozyme is producible that is substantially complementary to at least a portion of a SOCS-1 transcript.

6. The method of claim 1 or claim 2, wherein the expression of SOCS-1 or the level and/or functional activity of the SOCS-1 expression product is reduced or abrogated using a construct from which an RNAi molecule is producible that is interactive with a SOCS-1 transcript.

7. The method of claim 1 or claim 2, wherein the mammary cell is a mammary epithelial cell.

8. The method of claim 1 or claim 2, wherein the mammary cell is a mammary ductal epithelial cell.

9. A method for modulating mammopoiesis, comprising modulating in a mammary cell the expression of a gene or the level and/or functional activity of an expression product of the gene, wherein the gene is selected from SOCS-1 or a gene belonging to the same regulatory or biosynthetic pathway as SOCS-1.

10. A method for modulating mammopoiesis, comprising modulating in a mammary cell the expression of SOCS-1 or the level and/or functional activity of an expression product of SOCS-1.

11. The method of claim 9 or claim 10, wherein the expression of SOCS-1 or the level and/or functional activity of the SOCS-1 expression product is reduced or abrogated.

12. The method of claim 9 or claim 10, wherein the expression of SOCS-1 or the level and/or functional activity of the SOCS-1 expression product is reduced or abrogated using a construct from which an antisense molecule is producible that is substantially complementary to at least a portion of SOCS-1.

13. The method of claim 9 or claim 10, wherein the expression of SOCS-1 or the level and/or functional activity of the SOCS-1 expression product is reduced or abrogated using a construct from which a ribozyme is producible that is substantially complementary to at least a portion of a SOCS-1 transcript.

14. The method of claim 9 or claim 10, wherein the expression of SOCS-1 or the level and/or functional activity of the SOCS-1 expression product is reduced or abrogated using a construct from which an RNAi molecule is producible that is interactive with a SOCS-1 transcript.

15. The method of claim 9 or claim 10, wherein the mammary cell is a mammary epithelial cell.

16. The method of claim 9 or claim 10, wherein the mammary cell is a mammary ductal epithelial cell.

17. A method for modulating the differentiation and/or expansion of the lobuloalveolar system, comprising modulating the expression of a gene or the level and/or functional activity of an expression product of the gene in a mammary cell, wherein the gene is selected from SOCS-1 or a gene belonging to the same regulatory or biosynthetic pathway as SOCS-1.

18. A method for modulating the differentiation and/or expansion of the lobuloalveolar system, comprising modulating in a mammary cell the expression of SOCS-1 or the level and/or functional activity of an expression product of SOCS-1.

19. The method of claim 17 or claim 18, wherein the expression of SOCS-1 or the level and/or functional activity of the SOCS-1 expression product is reduced or abrogated.

20. The method of claim 17 or claim 18, wherein the expression of SOCS-1 or the level and/or functional activity of the SOCS-1 expression product is reduced or abrogated using a construct from which an antisense molecule is producible that is substantially complementary to at least a portion of SOCS-1.

21. The method of claim 17 or claim 18, wherein the expression of SOCS-1 or the level and/or functional activity of the SOCS-1 expression product is reduced or abrogated using a construct from which a ribozyme is producible that is substantially complementary to at least a portion of a SOCS-1 transcript.

22. The method of claim 17 or claim 18, wherein the expression of SOCS-1 or the level and/or functional activity of the SOCS-1 expression product is reduced or abrogated using a construct from which an RNAi molecule is producible that is interactive with a SOCS-1 transcript.

23. The method of claim 17 or claim 18, wherein the mammary cell is a mammary epithelial cell.

24. The method of claim 17 or claim 18, wherein the mammary cell is a mammary ductal epithelial cell.

25. A method for modulating lactogenesis, comprising modulating the expression of a gene or the level and/or functional activity of an expression product of the gene in a mammary cell, wherein the gene is selected from SOCS-1 or a gene belonging to the same regulatory or biosynthetic pathway as SOCS-1.

26. A method for modulating lactogenesis, comprising modulating in a mammary cell the expression of SOCS-1 or the level and/or functional activity of an expression product of SOCS-1.

27. The method of claim 25 or claim 26, wherein the expression of SOCS-1 or the level and/or functional activity of the SOCS-1 expression product is reduced or abrogated.

28. The method of claim 25 or claim 26, wherein the expression of SOCS-1 or the level and/or functional activity of the SOCS-1 expression product is reduced or abrogated using a construct from which an antisense molecule is producible that is substantially complementary to at least a portion of SOCS-1.

29. The method of claim 25 or claim 26, wherein the expression of SOCS-1 or the level and/or functional activity of the SOCS-1 expression product is reduced or abrogated using a construct from which a ribozyme is producible that is substantially complementary to at least a portion of a SOCS-1 transcript.

30. The method of claim 25 or claim 26, wherein the expression of SOCS-1 or the level and/or functional activity of the SOCS-1 expression product is reduced or abrogated using a construct from which an RNAi molecule is producible that is interactive with a SOCS-1 transcript.

31. The method of claim 25 or claim 26, wherein the mammary cell is a mammary epithelial cell.

32. The method of claim 25 or claim 26, wherein the mammary cell is a mammary ductal epithelial cell.

33. The method of claim 1, wherein the mammary cell is associated with the reproductive system of a mammal, wherein proliferation of said mammary cell results in tumorigenesis, which can be modulated by contacting the mammary cell with an agent for a time and under conditions sufficient to modulate the expression of a gene or the level or functional activity of an expression product of the gene.

34. The method of claim 1, wherein the mammary cell is associated with the reproductive system of a mammal, wherein proliferation of said mammary cell results in tumorigenesis, which can be modulated by modulating the expression of SOCS-1 or the level or functional activity of an expression product of SOCS-1.

35. The method of claim 33 or claim 34, wherein the expression of SOCS-1 or the level and/or functional activity of the SOCS-1 expression product is enhanced.

36. The method of claim 33 or claim 34, wherein the expression of SOCS-1 or the level and/or functional activity of the SOCS-1 expression product is enhanced using a construct comprising a nucleic acid molecule which is selected from the group consisting of a SOCS-1 polynucleotide, a biologically active fragment of a SOCS-1 polynucleotide, a variant of a SOCS-1 polynucleotide and a variant of a biologically active fragment of a SOCS-1 polynucleotide, and which is operably connected to a transcriptional control element.

37. The method of claim 33 or claim 34, wherein the expression of SOCS-1 or the level and/or functional activity of the SOCS-1 expression product is enhanced using a proteinaceous molecule selected from the group consisting of a SOCS-1 polypeptide, a biologically active fragment of a SOCS-1 polypeptide, a variant of a SOCS-1 polypeptide, a variant of a biologically active fragment of a SOCS-1 polypeptide, a derivative of a SOCS-1 polypeptide and a derivative of a biologically active fragment of a SOCS-1 polypeptide.

38. The method of claim 33 or claim 34, wherein the expression of SOCS-1 or the level and/or functional activity of the SOCS-1 expression product is enhanced using a proteinaceous molecule comprising an SH2 domain of a SOCS-1 polypeptide.

39. The method of claim 33 or claim 34, wherein the expression of SOCS-1 or the level and/or functional activity of the SOCS-1 expression product is enhanced using a proteinaceous molecule comprising a SOCS box of a SOCS-1 polypeptide.

40. The method of claim 33 or claim 34, wherein the expression of SOCS-1 or the level and/or functional activity of the SOCS-1 expression product is enhanced using a proteinaceous molecule comprising an SH2 domain of a SOCS-1 polypeptide and a SOCS box of a SOCS-1 polypeptide.

41. The method of claim 33 or claim 34, wherein the cell is a cell of a reproductive organ selected from the group consisting of breast, ovary, endometrium, testes, and prostate.

42. The method of claim 33 or claim 34, wherein the cell is a mammary cell.

43. The method of claim 33 or claim 34, wherein the cell is an ovarian cell.


SEQUENCE LISTING (14 sequences)

SEQ ID NO: 1 - DNA, 908 bp, Homo sapiens
  CDS (14)..(649): Human SOCS-1 CDS
ccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacc

SEQ ID NO: 2 - protein, 211 aa, Homo sapiens
MVAHNQVAADNAVSTAAEPRRRPEPSSSSSSSPAAPARPRPCPAVPAPAPGDTHFRTFRSHADYRRITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRRVRPLQELCRQRIVATVGRENLARIPLNPVLRDYLSSFPFQI

SEQ ID NO: 3 - DNA, 1193 bp, Mus musculus
  CDS (130)..(768): Mouse SOCS-1 CDS
ggcacggctcccagccccggagcatgcgcgacagccgccccggagcccccagccgcggctccccgcgtcctgccgccagcgcagccccggacgctatggcccacccctccagctggcccctcgagtaggatggtagcacgcaaccaggtggcagccgacaatgcgatctccccggcagcagagccccgacggcggtcagagccctcctcgtcctcgtcttcgtcctcgccagcggcccccgtgcgtccccggccctgcccggcggtcccagccccagcccctggcgacactcacttccgcaccttccgctcccactccgattaccggcgcatcacgcggaccagcgcgctcctggacgcctgcggcttctattggggacccctgagcgtgcacggggcgcacgagcggctgcgtgccgagcccgtgggcaccttcttggtgcgcgacagtcgccaacggaactgcttcttcgcgctcagcgtgaagatggcttcgggccccacgagcatccgcgtgcacttccaggccggccgcttccacttggacggcagccgcgagaccttcgactgccttttcgagctgctggagcactacgtggcggcgccgcgccgcatgttgggggccccgctgcgccagcgccgcgtgcggccgctgcaggagctgtgtcgccagcgcatcgtggccgccgtgggtcgcgagaacctggcgcgcatccctcttaacccggtactccgtgactacctgagttccttccccttccagatctgaccggctgccgctgtgccgcagcattaagtgggggcgccttattatttcttattattaattattattatttttctggaaccacgtgggagccctccccgcctgggtcggagggagtggttgtggagggtgagatgcctcccacttctggctggagacctcatcccacctctcaggggtgggggtgctcccctcctggtgctccctccgggtcccccctggttgtagcagcttgtgtctggggccaggacctgaattccactcctacctctccatgtttacatattcccagtatctttgcacaaaccaggggtcggggagggtctctggcttcatttttctgctgtgcagaatatcctattttatatttttacagccagtttaggtaataaactttattatgaaagtttttttttaaaagaaacaaa

SEQ ID NO: 4 - protein, 212 aa, Mus musculus
MVARNQVAADNAISPAAEPRRRSEPSSSSSSSSPAAPVRPRPCPAVPAPAPGDTHFRTFRSHSDYRRITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRRVRPLQELCRQRIVAAVGRENLARIPLNPVLRDYLSSFPFQI

SEQ ID NO: 5 - protein, 63 aa, Homo sapiens
  DOMAIN (1)..(63): Human SOCS-1 SH2 domain
GFYWGPLSVHGAHERLRAEPVGTFLVRDSRQRNCFFALSVKMASGPTSIRVHFQAGRFHLDGS

SEQ ID NO: 6 - protein, 63 aa, Mus musculus; identical to SEQ ID NO: 5
  DOMAIN (1)..(63): Mouse SOCS-1 SH2 domain

SEQ ID NO: 7 - protein, 104 aa, Homo sapiens
  DOMAIN (1)..(104): Human SOCS-1 extended SH2 domain
ITRASALLDACGFYWGPLSVHGAHERLRAEPVGTFLVRDSRQRNCFFALSVKMASGPTSIRVHFQAGRFHLDGSRESFDCLFELLEHYVAAPRRMLGAPLRQRR

SEQ ID NO: 8 - protein, 104 aa, Mus musculus
  DOMAIN (1)..(104): Mouse SOCS-1 extended SH2 domain
ITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRR

SEQ ID NO: 9 - protein, 34 aa, Homo sapiens
  DOMAIN (1)..(34): Human SOCS-1 SOCS box
VRPLQELCRQRIVATVGRENLARIPLNPVLRDYL

SEQ ID NO: 10 - protein, 34 aa, Mus musculus
  DOMAIN (1)..(34): Mouse SOCS-1 SOCS box
VRPLQELCRQRIVAAVGRENLARIPLNPVLRDYL

SEQ ID NO: 11 - protein, 43 aa, Homo sapiens
  DOMAIN (1)..(43): Human SOCS-1 extended SOCS box
PLRQRRVRPLQELCRQRIVATVGRENLARIPLNPVLRDYLSSF

SEQ ID NO: 12 - protein, 43 aa, Mus musculus
  DOMAIN (1)..(43): Mouse SOCS-1 extended SOCS box
PLRQRRVRPLQELCRQRIVAAVGRENLARIPLNPVLRDYLSSF

SEQ ID NO: 13 - DNA, 25 bp, Artificial Sequence
  Beta-casein sense primer
atgaaggtcttcatcctcgcctgcc

SEQ ID NO: 14 - DNA, 26 bp, Artificial Sequence
  Beta-casein antisense primer
gctggaccagagactgaggaaggtgc